(19) United States
(12) Patent Application Publication
(10) Pub. No.: US 2023/0139404 A1
(43) Pub. Date: May 4, 2023

(54) Improved single-cell protein production using antioxidants

(71) Applicant: The Protein Brewery B.V., Breda (NL)

(72) Inventors: Kirsten Knobel, Breda (NL); Ap de Haan, Prinsenbeek (NL); Wilhelmus Theodorus Antonius Maria de Laat, Breda (NL)

(73) Assignee: The Protein Brewery B.V., Breda (NL)

(21) Appl. No.: 17/908,543

(22) PCT Filed: Mar. 5, 2021
(86) PCT No.: PCT/EP2021/055550

§ 371 Date: Sep. 1, 2022

(30) Foreign Application Priority Data
Mar. 6, 2020 (EP) 20161606.7

(51) Int. Cl. A23J 3/20; A23J 1/00; A23J 3/22; C12N 1/14

(52) U.S. Cl. CPC A23J 3/20; A23J 1/008; A23J 3/227; C12N 1/14

(57) ABSTRACT

The invention relates to a method for preparing a stable composition of single-cell protein by using antioxidants. The single-cell protein can be derived from a fungal biomass, wherein the biomass is pasteurised after antioxidants have been added to it. The stable composition has low TOTOX values and is suitable for use as a source of single-cell proteins in food or feed products. The invention also relates to the pasteurised product, and to food products comprising it.

### FIELD OF THE INVENTION

The invention relates to a method for preparing a stable composition of single-cell protein by using antioxidants. The single-cell protein can be derived from a fungal biomass, wherein the biomass is pasteurised after antioxidants have been added to it. The stable composition has low TOTOX values and is suitable for use as a source of single-cell proteins in food or feed products. The invention also relates to the pasteurised product, and to food products comprising it.

### BACKGROUND ART

Single-cell proteins (SCP) are an interesting alternative to meat proteins and as protein source in many other food applications such as breakfast cereals, bread, pasta, dairy, ice cream, chocolate, and soups. When fungal SCP is produced, it can be produced from sugar rich crops in a more sustainable way as compared to the production of meat protein, because SCP yields many more tonnes of protein per hectare compared to meat production and has lower nitrogen emission. One method for producing protein-rich food or animal feed is to produce SCP by means of fermentation (Suman et al., 2015, Int J. Curr. Microbiol. Appl. Sci., Vol 4, No 9, pp 251-262). Fermentation in this respect is understood as the microbial conversion of carbohydrate-rich feedstocks into protein-rich products consisting of microbial cells such as bacteria, yeasts or fungi. The use of SCP as animal feed and food ingredient brings the further advantages that microbial cells have a high content of essential amino acids and that microbial cells, e.g. when applied to supplement grain-based diets, produce useful enzymes such as phytase, xylanases, pectinases, proteases, cellulases, amylases, all of which can have a positive effect on digestibility of the compound feeds that have high contents of e.g. the anti-nutritional compound phytate, poorly digestible fibres etc. Furthermore, in particular fungal cells can be very rich in trace elements and vitamins making the fermented feedstuffs very nutritive.

SCP is already used in food products for human consumption. For instance, Quorn™ comprises a mycoprotein produced by Fusarium venenatum which contains Vitamin B1 (Thiamin), Vitamin B2 (Riboflavin), Vitamin B3 (Niacin), Vitamin B5 (Pantothenic acid), and Biotin. One problem in SCP production is the concentration of the SCP-biomass that is produced in the fermentation broth, particularly in the case of submerged fermentations with bacteria or yeasts. Another problem is the need for expensive enzymes to convert the cheap polymeric carbon sources to monomeric fermentable sugars. Furthermore, to avoid infection when using mesophilic microorganisms for SCP production sterile fermentation conditions need be applied, which leads to prohibitive operational costs due to high capital investments and energy demands (WO2018/029353). Some of these issues have been addressed by using solid state fermentation with thermophilic fungi (WO2018/029353). WO2018/029353 describes SCP derived from fungi. The SCP is easily harvested and pressed in a sieving process.

U.S. Pat. No. 8,481,295B2 discloses the production of thermophilic fungi as animal feed ingredient using batch fermentation on thin stillage from ethanol refineries. However, the fungal strain used therein does not perform well at pH<4 and temperature higher than 45° C., which makes the process sensitive to bacterial and yeast contamination.

Gregory K. F et al. (1977, Anim. Feed Sci. Technol. 2:7-19) disclose attempts to use thermotolerant fungi for the conversion of cassava, in the course of which many thermotolerant fungi were isolated. However, these attempt did not lead to commercialized products as contamination issues remained with their organisms, or undesirable human pathogens were used (e.g. Aspergillus fumigatus), while their Mucor strains were found to be poorly digestible in rat studies. As described in WO2018/029353, several sources have also reported studies involving the thermotolerant fungus Cephalosporium eichornia for production of SCP.

For improving storage stability, SCP can be cooled, frozen or dried. However, when the SCP is sold as an ingredient in the food industry, the microorganism might continue or recommence growth in the final food product. Therefore, SCP for use in food products generally need a reduction of germ counts to <10 fungi/gr, which is a typical food norm. Such reduction of germ count is conveniently achieved via pasteurisation.

Pasteurisation equipment is well known and commercially available in many shapes and forms. Because it is a form of heat treatment, pasteurisation involves intensive energy consumption. When pasteurisation is to be done after for example submerged fermentation of the single celled organism, the volume to be treated is rather high as e.g. fungal biomass will not generally reach a biomass dry matter content over 5% dry matter. This is because during such fermentation conditions the oxygen transfer is generally limiting as a result of the viscous behaviour of the broth. For this reason, fermentation broths are sometimes purposely diluted even further.

For context, a target of milk pasteurisation is to achieve 99.999% (5-log) reduction in viable microorganisms. Pasteurisation is generally achieved with high-temperature-short-time equipment, which uses continuous heat processing. The temperature-time combinations common for milk pasteurisation have been selected to optimize microbial kill while minimizing the impact on the nutritional quality of milk. Pasteurised milk (heated to 60° C. for about 20 minutes) must be stored under refrigeration and has a relatively short shelf life. In contrast, ultrapasteurised (UP) milk (heated to 125-138° C. for 2-4 seconds) can be stored for up to 3 months under refrigeration. Milk subjected to or ultra-high temperature (UHT) treatment (heated to 135-140° C. for a few seconds) can be stored for 3-6 months at ambient temperatures. However, studies with consumer panels have revealed that many consumers can distinguish between pasteurised and UHT milk by taste. Consumers often prefer pasteurised milk because of the flavour, describing UHT milk as having a ‘cooked’ flavour (L. Meunier-Goddik, S. Sandra, Encyclopedia of Dairy Sciences, 2011, 274-280). High temperature treatment has also been associated with adverse effects on the taste of foodstuff containing Mucorales fungi (WO0167886).

Off-flavours in SCP are undesirable. Off-flavours are often the result of lipid oxidation, and therefore are also associated with the total oxidative load to which a fat has been exposed. A value representing this oxidative load is the TOTOX value, an abbreviation for total oxidation value. It is a dimensionless number used to describe the total oxidative load to which a fat has been exposed. The value is calculated as the sum of the anisidine value and twice the peroxide value. Anisidine value (AnV) is a measure of aldehyde production during oxidation of fats. The value expresses the absorbance of fat reacted with p-anisidine, trivial name for p-methoxyaniline, under specified conditions. It is used to characterize the oxidative history of fat because aldehydes normally originate from oxidation of unsaturated fatty acids. Peroxide value (PV) is a measure of the extent of oxidation of a fat or oil. The value indicates the quantity of oxidized substances, normally hydroperoxides, that liberate iodine from potassium iodide under specified conditions. PV is expressed in milliequivalents of active oxygen per kg fat (Beare-Rogers et al., Lexicon of lipid nutrition (IUPAC Technical Report), 2009 DOI: 10.1351/pac200173040685). Oils with a TOTOX value under 80 have been described as having a good flavour evaluation (JP2014054248).

It is an object of the invention to improve shelf life of a single-cell protein. It is an object of the invention to provide a single-cell protein with a low TOTOX value, preferably with a consistently low TOTOX value. It is an object of the invention to improve pasteurisation of single-cell protein biomass, to reduce the energy requirement of pasteurisation, to reduce the complexity of required equipment and fermentation setups for production of pasteurised SCP. It is an object of the invention to produce (pasteurised) SCP with improved organoleptic qualities such as improved taste, smell, and/or mouth feel, preferably while improving or maintaining stability or shelf life.

### SUMMARY OF THE INVENTION

The invention provides a method for producing a biomass composition comprising an antioxidant and biomass, comprising the steps of:
i) providing a biomass;
ii) adding an antioxidant to the biomass to obtain a biomass mixture;
iii) pasteurising the biomass mixture to obtain the biomass composition; and
iv) optionally washing the biomass composition.
   Preferably the antioxidant is at least one of a free radical scavenger, such as carnosol; carnosic acid; rosemary extract; a flavan-3-ol such as epicatechin, epigallocatechin, epigallocatechin gallate (EGCG), epicatechin gallate; a flavonoid such as kaemferol, quercetin, or myricetin; green tea extract; butylated antioxidants such as butylated hydroxytoluene (BHT), butylated hydroxyanisole, or tert-butylhydroquinone (TBHQ); gallic acid; a gallate ester such as propyl gallate; carvone; a caffeate ester such as methyl caffeate, ethyl caffeate, caffeic acid phenethyl ester (CAPE), or rosmarinic acid; spearmint extract; a vitamin E such as a tocopherol or a tocotrienol; a vitamin A such as retinol, retinoic acid, or retinal; a provitamin A such as a carotenoid, β-carotene, lutein, zeaxanthin, or echinenone; a quinolone-based antioxidant such as ethoxyquin; or seaweed extract; or an oxygen reducer, such as ascorbic acid or acerola extract; or a chelator, such as citric acid or EDTA. Preferably the obtained biomass composition has a TOTOX value of under 50, preferably under 35, more preferably under 20, and wherein the TOTOX value preferably remains under 50, more preferably under 40, for at least 6 weeks. In some embodiments the biomass is derived from a fermentation broth, preferably from a submerged fermentation process, wherein the fermentation broth preferably comprises a biomass dry matter content of at most 6%, more preferably of at most 4%, or wherein the fermentation broth is sieved to obtain the biomass, preferably using a sieving belt, a vibrating sieve, or a screen sieve. In some embodiments the biomass comprises at most 500 mg/kg dry matter iron, preferably at most 300 mg/kg dry matter, more preferably at most 150 mg/kg dry matter, most preferably at most 50 mg/kg dry matter. Preferably, the pH of the biomass in step iii) is at most 4.5, preferably at most 3.5. In some embodiments, the biomass comprises at least 7% dry matter, preferably at least 10% dry matter. Preferably, the biomass is a biomass derived from a fungal strain, wherein the fungal strain is preferably a strain of a fungal genus selected from the group consisting of Rasamsonia, Talaromyces, Penicillium, Acremonium, Humicola, Paecilomyces, Chaetomium, Rhizomucor, Rhizopus, Thermomyces, Myceliophthora, Thermoascus, Thielavia, Mucor, Stibella, Melanocarpus, Malbranchea, Dactylomyces, Canariomyces, Scytalidium, Myriococcum, Corynascus, and Coonemeria, more preferably the genus is Rhizomucor, preferably the strain is Rhizomucor pusillus, more preferably Rhizomucor pusillus strain CBS 143028, or a strain that is a single colony isolate or a derivative thereof.

In preferred embodiments the pasteurisation is performed in an in-line heating unit that preferably comprises a pipe heater, a heating block, or a steam infusion element, more preferably a steam infusion element, and wherein the in-line heating unit optionally comprises a mixing element such as a static mixer. In some embodiments the pasteurisation is performed for at most 45 minutes, preferably for at most 5 minutes, more preferably from about 0.5 to about 3 minutes, most preferably from about 1 to 2 minutes. Preferably, pasteurisation is performed at a temperature of at least 70° C., preferably at least 74° C., more preferably at least 80° C., most preferably at least 86° C. Preferably antioxidant is only added during step ii).

The invention also provides a biomass composition comprising an antioxidant and biomass, obtainable by a method as described above, preferably comprising from about 6% to 12% lipids and from about 35% to 55% proteins, based on dry weight. The invention also provides a food or feed product comprising that biomass composition. The invention also provides the use of that biomass composition in the manufacture of a food product, or as a source of at least one of fiber or protein, for use in the manufacture of a food product.

### DESCRIPTION OF EMBODIMENTS

The inventors have surprisingly found that single-cell protein that has been mixed with antioxidant prior to pasteurisation has superior TOTOX values and is more pleasing to the senses. Accordingly the invention provides a method for producing a biomass composition comprising an antioxidant and biomass, comprising the steps of:
i) providing a biomass;
ii) adding an antioxidant to the biomass to obtain a biomass mixture;
iii) pasteurising the biomass mixture to obtain the biomass composition; and
iv) optionally washing the biomass composition.

Such a method is referred to hereinafter as a method or process according to the invention.

The inventors have also surprisingly found that single-cell protein that has been pasteurised for at most 45 minutes at a temperature of at least 70° C. is both adequately low in germ count, as well as more pleasant to the senses. The invention encompasses the method as well as products obtained thereby, and uses of such products. Accordingly, the invention provides a method for pasteurising a biomass, comprising the steps of:
i) providing a biomass;
iii) pasteurising the biomass to obtain pasteurised biomass, wherein the biomass is pasteurised for at most 45 minutes at a temperature of at least 70° C., wherein this is preferably performed by streaming the biomass through an in-line heating unit to obtain pasteurised biomass, wherein the biomass has a residence time in the heating unit of at most 45 minutes, and wherein the heating unit has a temperature of at least 70° C., and
iv) optionally recovering the pasteurised biomass.

The term “single-cell protein” will be abbreviated “SCP” and is herein understood to refers to biomass consisting essentially of cells of organisms that exist in unicellular, or single cell, state, including unicellular bacteria, yeasts, fungi, or algae, yet also encompassing microorganisms that grow in filamentous form (hypha), and which biomass, preferably in dried form, is suitable as dietary source of protein or protein supplement in human food or in animal feed.

### Method Step i) Provision of a Biomass

In step i) a biomass is provided. This biomass is preferably a biomass of unicellular or filamentous organisms, of which bacteria, yeasts, fungi, or algae are preferred. In some embodiments the biomass is a unicellular biomass. In other embodiments the biomass is of a filamentous organism. In preferred embodiments, the biomass that is provided is a biomass that is derived from a fungal strain, preferably a filamentous fungal strain. It is preferably not a pasteurised biomass, but derived from a fermentation without having been heat treated.

The fungus that is used in the method of the invention, i.e. the fungus that is grown in the process, preferably is a thermophilic fungus. A thermophilic fungus for use in the invention preferably is a fungus that grows at a temperature of at least 45, 46, 47, 48, 50, 51, 52, or 55° C., sometimes even higher than 56° C. A thermophilic fungus for use in the invention preferably is also a fungus that grows at low, i.e. acidic pH. A preferred thermophilic fungus grows at a pH of 3.8, 3.75, 3.74, 3.73, 3.72, 3.71, 3.6, 3.5, 3.4, 3.3, 3.2, 3.1, 3.0, 2.9, 2.8, 2.7, 2.6, 2.5 or less, more preferably 3 or less. Preferably the fungus is grown at that pH. A thermophilic fungus for use in the invention preferably is a cellulolytic and/or hemi-cellulolytic fungus.

“Fungi” are herein defined as eukaryotic microorganisms and include all species of the subdivision Eumycotina (Alexopoulos et al., 1962, In: Introductory Mycology, John Wiley & Sons, Inc., New York). The term fungus thus includes both filamentous fungi and yeast. “Filamentous fungi” are herein defined as eukaryotic microorganisms that include all filamentous forms of the subdivision Eumycotina and Oomycota (as defined by Hawksworth et al., 1983, In: Ainsworth and Brisby's Dictionary of the Fungi. 7^{th} ed. Commonwealth Mycological Institute, Kew, Surrey). The filamentous fungi are characterized by a mycelial wall composed of chitin, cellulose, glucan, chitosan, mannan, and other complex polysaccharides. Vegetative growth is by hyphal elongation and carbon catabolism is obligately aerobic.

A thermophilic fungus for use in a method of the invention preferably is filamentous fungus. A preferred thermophilic fungus for use in the invention is a strain of a fungal genus selected from the group consisting of Rasamsonia, Talaromyces, Penicillium, Acremonium, Humicola, Paecilomyces, Chaetomium, Rhizomucor, Rhizopus, Thermomyces, Myceliophthora, Thermoascus, Thielavia, Mucor, Stibella, Melanocarpus, Malbranchea, Dactylomyces, Canariomyces, Scytalidium, Myriococcum, Corynascus, and Coonemeria. Preferably the genus is Rhizomucor, preferably the strain is Rhizomucor pusillus, more preferably Rhizomucor pusillus strain CBS 143028, or a strain that is a single colony isolate or a derivative thereof. More preferably, the thermophilic fungus is a strain of a fungal species selected from the group consisting of Rasamsonia composticola, Talaromyces emersonii, Rasamsonia emersonii, Thermomucor indicae-seudaticae, Rhizomucor miehei, Rhizomucor pusillus, Thielavia terricola var minor, a Rhizopus sp. and Thermoascus thermophilus. Suitable strains of these thermophilic fungi can e.g. be isolated from Dutch compost and have been successfully used by the inventors to demonstrate that these thermophilic fungi grow well on complex nutrients at high temperature and low pH. Next to this, many thermophylic strains are suitable for use in the invention such as Thielavia terricola var minor and Thermoascus thermophilus, which also grow well at high temperature and low pH. A Rhizopus sp. can be any one of Rhizopus oryzae, Rhizopus chlamydosporus, Rhizopus microsporus, Rhizopus stolonifer or Mucor indicus. Alternatively, a Rhizopus sp. can be a yet unidentified Rhizopus or Mucor species that corresponds with Rhizopus sp. CBS 143160. Preferably the Rhizopus sp. is safe for use in food, more preferably the Rhizopus sp. is a tempeh starter.

Preferred strains of the above-mentioned thermophilic fungi for use in the invention include the following strains that were deposited under the regulations of the Budapest Treaty at the Westerdijk Fungal Biodiversity Institute Utrecht, The Netherlands (formerly referred to as Centraalbureau voor Schimmelcultures, CBS) at the dates indicated and assigned the accession numbers as indicated: Rasamsonia composticola CBS 141695 (Jul. 29, 2016), Thermomucor indicae-seudaticae CBS 143027 (Jul. 21, 2017), Rhizomucor miehei CBS 143029 (Jul. 21, 2017), Rhizomucor pusillus CBS 143028 (Jul. 21, 2017), Rasamsonia emersonii strain CBS 143030 (Jul. 30, 2017) and Rhizopus sp. CBS 143160 (Aug. 11, 2017). Further preferred strains for use in the invention include Thermomucor indicae-seudaticae CBS 104.75, Thermoascus thermophilus CBS 528.71, Thielavia terrestris CBS 546.86, Talaromyces emersonii CBS 393.64 and Thermothelomyces thermophila CBS 117.65. Particularly preferred for use in the invention are the strains Rasamsonia composticola strain CBS 141695, Rasamsonia emersonii CBS 143030, Thermomucor indicae-seudaticae CBS 143027, Rhizomucor miehei CBS 143029, Rhizopus sp. CBS 143160 and Rhizomucor pusillus CBS 143028.

A thermophilic fungus for use in the invention further preferably is a fungus from which is biomass can be obtained with a high protein content. Preferably the protein content of the biomass is at least 30, 35, 40, 45, 50 or 55% (w/v) on dry matter basis. The high protein strains most likely have a lower content of carbon reserve and/or storage compounds, such as e.g. trehalose, glycogen and/or lipids.

A thermophilic fungus for use in the invention further preferably is a fungus of which the proteins in the biomass contain one or more of the essential amino acids. Preferably the proteins are rich in such essential amino acids. Essential amino acids are herein understood to include at least one or more of lysine, phenylalanine, threonine, methionine, valine, arginine, histidine, tryptophan, isoleucine and leucine, of which, lysine, threonine, methionine are most preferred.

As the SCP product is intended for use in food or feed for animals for human consumption, the production of mycotoxins, such as e.g. Ochratoxin A and Fumonisins, by the thermophilic fungus to be applied is undesirable. Therefore, a thermophilic fungus for use in the invention preferably is selected that does not produce any mycotoxins. This screening is preferably done by genetic means, by verifying e.g. with PCR or with whole genome sequencing, the absence of the presence of genes in mycotoxin pathways, and in the case such genes are present, by verifying that, under process conditions used, these genes are not expressed and/or these toxic compounds are not produced.

The microorganism comprised in the biomass preferably produces its own extracellular hydrolytic enzymes, which allow the process to be run under non-sterile conditions because at temperature higher than 45° C. and a pH of less than 3.8 other (micro)organisms will not be able to invade and/or compete with the microorganism of interest, preferably a fungus, more preferably a thermophilic fungus. Therefore, preferably a thermophilic fungus is used that can grow on energy-rich carbon-dominated feedstocks including both simple sugars such as sucrose and glucose, fructose, as well as polymeric sugars such as starch, inuline, cellulose, hemicellulose, chitin, pectin as well as organic acids such as lactic acid, acetic acid, formic acid, and ethanol and methanol (these metabolites are often formed in silage processes or from splitting them off from pectin and hemicellulose), as well as lipids present in the form of a triglyceride or phospholipids. Also the conversion of other sugars such as those present in hemicellulose; rhamnose, fucose, galactose, xylose arabinose, mannose, galacturonic acid, glucuronic acid etc. is needed as well as raffinose, melibiose, stachyose etc. is preferred to enhance the protein product of the feed ingredient and minimizing carbon burden from the filtrate which has to go to the waste water treatment/biogas installation. Also, the conversion of betaine, ferulic acid and coumaric acid by the fungus is preferred to maximize yield. The advantage of the many thermophilic fungi that occur in processes like composting is that they can stand very harsh conditions and can produce the enzymes to split the polymeric substrates such as carbohydrates into monomeric sugars and convert them.

Alternatively, a thermophilic fungus to be used in the processes of the invention is genetically modified to produce increased amounts of hydrolytic enzymes, preferably invertase (e.g. for Rasamsonia), cellulolytic and/or lignocellulolytic enzymes, are used in the present invention, such as e.g. described in WO2011/000949. The enhanced enzyme production can lead to reduced hydrolysis times, smaller tanks can then be used and the enzyme-containing filtrate/decantate can be commercialised as secondary product.

The biomass is preferably derived from a fermentation broth, more preferably from a submerged fermentation process. Provision of a biomass preferably comprises the step of: i-a) growing a thermophilic fungus in a medium containing a fermentable carbon-rich feedstock. Preferably, in step i-a) the fungus is grown in submerged culture. Preferably, in step i-a) the fungus is grown under non-sterile conditions. Preferably, in step i-a) the fungus is grown at a temperature of 46, 47, 48, 49, 50, 51, 52, 53, 54 or 55° C. or more. Preferably, in step i-a) the fungus is grown at a pH of 3.8, 3.75, 3.74, 3.73, 3.72, 3.71, 3.7, 3.6, 3.5, 3.4, 3.3, 3.2, 3.1, 3.0, 2.9, 2.8, 2.7, 2.6, 2.5 or less. The process preferably comprises a further step of i-b) recovery of microorganism from the medium in the form of (concentrated) biomass of the thermophilic fungus grown in step i-a). The biomass that is to be pasteurised preferably has a high cell density.

As is known from WO2018/029353, one problem to be solved in the production of SCP at low pH is the toxicity of many fermentable carbon-rich feedstocks. Particularly hydrolysed biomass or silage products are likely to contain compounds that are toxic to most microorganism, including e.g. organic acids such as acetic acid, lactic acid, ferulic acid, coumaric acid, formic acid. These acids are especially toxic at low pH, when they are in non-dissociated form and as such can readily penetrate the cell wall and acidify the cell's interior. When such fermentable carbon-rich feedstocks are applied in fermentations at low pH and at a feedstock dry matter concentration (w/v) that is higher than 2, 5 or 10%, the toxicity will be prohibitive for fungal growth.

Therefore, preferably in a process of the invention, in step i-a), the concentration of the carbon-rich feedstock is at a level at which toxic compounds in the feedstock do not reduce the growth rate of the fungus. More preferably, the carbon-rich feedstock is fed to the medium at a rate at which at which toxic compounds in the feedstock do not reduce the growth rate of the fungus. For example, in a process according to the invention, the carbon-rich feedstock in the medium is at a concentration, or is fed to the medium at a rate to maintain a concentration, of less than 5, 4, 3 or 2% (w/v) dry matter. Dry matter in feedstock is generally the substrate for fermentation and is distinct from the biomass dry matter that is a measure for the amount of microorganism in the a broth or biomass. Feedstock dry matter is preferably present as 1-20% or 2-20% dry matter (w/v), more preferably as 2-15%, still more preferably at 2-10%, still more preferably at 2-6%. In preferred embodiments, the fermentation broth comprises a biomass dry matter content in the range of 0.5-7%, preferably of at most 6%, preferably of at most 5%, more preferably of at most 4%. Even more preferably it is at most 3%, still more preferably at most 2%, still more preferably at most 1.5%, most preferably at most 1%. This dry matter content is preferably at least 0.2%, more preferably at least 0.5%, still more preferably at least 1%, even more preferably at least 2%, still more preferably at least 3%, most preferably at least 3.5%. A higher biomass dry matter content reduces the volume that needs to be pasteurised, a lower biomass dry matter content improves fermentation conditions such as aeration and mixing.

The fermentability of a feedstock can conveniently be checked or monitored by measuring at least one of the CO₂ content and the O₂ content of the exhaust gas of the fermenter. The maximum concentration at which a feedstock can be used without negatively affecting the growth rate of the fungus can thus be determined by increasing concentration if the feedstock in the medium until a concentration is reached at which at least one of rate of CO₂ production and the rate of oxygen consumption decreases. Preferably therefore, in a process of to the invention, the concentration at which toxic compounds in the feedstock do not reduce the growth rate of the fungus is determined and/or defined as the highest concentration of the carbon-rich feedstock which does not cause a reduction in at least one of the rate of CO₂ production and the rate of O₂ consumption by the fungus. A well fermenting feedstock will allow a rapid increase of the rate of CO₂ production or oxygen consumption as may be determined by resp. an increase in CO₂ concentration or a decrease in the oxygen concentration in the off gas from the fermentation. When CO₂ evolution rate is low (and other factors such as nitrogen, carbon, minerals, and trace elements are not limiting), growth is slow and can be enhanced by diluting with water until growth starts taking off and CO₂ production accelerates.

WO2018/029353 discloses a fed-batch technique wherein the hydrolysed biomass is diluted to <2% dry matter before inoculation, followed by completion of the batch phase and, when organic acids and sugars are consumed, a start of a feed with hydrolysed biomass at a slow rate at glucose limiting conditions (e.g. glucose=<2 g/L) to allow the fungus to consume all the toxic organic acids fed to the fermenter. A preferred provided biomass for use in a method of the invention is therefore derived from a fed-batch process, a repeated fed-batch process (wherein repeatedly a part of the fermentation broth is harvested) or a continuous process. Preferably in such processes, the dilution rate, i.e. the rate at which the feedstock is fed into the fermenter, should be as high as possible but preferably not higher than the maximum specific growth rate of the fungus to prevent washing out of the fungus. The dilution rate preferably is in the range of 0.05 to 0.5, preferably to 0.4, more preferably to 0.2 1/hr, which refers to a residence time in the fermenter of 5 to 20 hours in the fermenter. The dilution rate thus preferably is at least 0.05 or 0.1 1/hr and preferably not higher than 0.4 or optionally than 0.2 1/hr.

A further preferred biomass is derived from a process that comprises the use of two or more fermenters, wherein at least a first fermenter is emptied for harvesting and optionally cleaning, while in at least a second fermenter growth of the fungus continues. Cleaning of the empty fermenter preferably comprises disinfection, e.g. by rinsing with acid (such as sulfuric acid or phosphoric acid), alkaline (such as NaOH or KOH), disinfectants (such as hydrogen peroxide or peracetic acid) or heat (e.g. steam), so as to control infection of the fermentation by e.g. bacteria or yeasts. Cleaning is preferably performed using a CIP installation. In one embodiment the biomass is derived from a process run in at least one pair of fermenters, which are alternatingly emptied for harvesting and optional cleaning once per 1, 2, 3, 4, or 5 days, or once per week, or once per month. This operation is an improved version of the process that allows non-sterile conditions to be practised without instability of the process or deviations in quality or process stability. In a further preferred embodiment of the process, after harvesting and optional cleaning, the empty first fermenter is filled with at least part of the content of the second fermenter wherein growth continued during harvesting and optional cleaning of the first fermenter. In a next round of the process for providing biomass, the second fermenter is harvested and optionally cleaned, and then filled with at least part of the content of the first fermenter wherein growth continued during harvesting and optional cleaning of the second fermenter, and so on. In yet another embodiment, the harvested fermentation batches are collected in a further continuous fermentation phase to allow higher product yields and/or stable feeding of the down-stream processing area.

It is preferred in the processes of the invention that for provision of the biomass the dry matter concentration (of the feedstock) is managed such that the oxygen consumption rate does not exceed the oxygen transfer capacity of the fermenter, which would lead to insufficient aeration and incomplete substrate consumption. Biomass can grow fast or slow. In preferred embodiments the biomass is a fast growing biomass. In other preferred embodiments the biomass is a slow growing biomass. Growth rate of the biomass is preferably controlled by increasing or decreasing the feed rate. Slow growing biomass has a lower RNA and protein content, and more fibers. Fast grown biomass has more protein and RNA and less fibers.

It is of note that the biomass that is pasteurised need not be identical to the fermentation mixture, as it can be further processed before pasteurisation. The dry matter concentration in the medium is further preferably optimized such that down-stream processing is most cost-efficient. To minimize the amount of harvested fermented medium to be pasteurised in step ii), the dry matter concentration of the provided biomass preferably is as high as possible. On the other hand, when the dry matter concentration of the feedstock in the medium is too high, the viscosity of the fungal broth will increase and the oxygen transfer will become problematic. The inventors have found that the optimal dry matter concentration of the feedstock in the medium in the fermenter is in the range of 1-20%, preferably 2-15% dry matter (w/v). Variations are possible, depending on the raw materials, salt stress, toxic metabolites, and type of fermenter used. The biomass dry matter content can also be at least 5%, preferably at least 6%. In preferred embodiments, the biomass comprises at least 7% dry matter, preferably at least 8%, more preferably at least 9%, still more preferably at least 10%, even more preferably at least 11%, most preferably at least 12% dry matter. This biomass dry matter content preferably relates to the content during pasteurisation. The dry matter content is preferably at most 15%, more preferably at most 13%, still more preferably at most 12%, still more preferably at most 11%, even more preferably at most about 10%. A preferred range is 5-15%, more preferably about 7-12%, still more preferably about 7-10%.

In addition the rheology of broth partly determined by the growth morphology of the fungus. The preferred growth morphology of the fungus in processes of the invention is a hyphal length that is short enough to give a low viscosity of the broth to allow easy oxygen transfer and mixing, but long enough to allow easy filtration or decantation at low g-values. Preferably therefore the hyphal length is in the range of 10-500 μm (micrometre) and preferably the hyphae are not too heavily branched. More preferably, the hyphal length is in the range of 30-300 μm. The mycelium preferably can be easily harvested by retention on a sieve or a screen, preferably with 0.1, 0.5, 1 or 2 mm diameter of pores.

It is further preferred that nitrogen limitation is avoided when biomass is fermented. The microorganism such as the fungus is therefore preferably grown under carbon-limitation. Thereby the protein content of the biomass produced can be maximised and accumulation of carbon reserve and/or storage compounds, such as e.g. trehalose, glycogen and/or lipids, as a result of carbon excess can be avoided.

A fermentable carbon-rich feedstock that can be used for provision of a biomass that is to be pasteurised in the process of the invention can be any feedstock that can serve as carbon and energy source for the thermophilic fungus. Such carbon-rich feedstock can be crops freshly harvested from the primary production of food sugars such as corn, sugar beet, thin juice, thick juice, sugar cane juice. However, particularly when the SCP is intended to be applied in animal feed, it is more logical and preferred to use as feedstock carbon-rich side- or by-products or waste streams from agriculture and/or food production, such as e.g. sugar beet pulp, liquid C-starch from grain processing, vegetable waste from production of pealed or cut vegetables or from rejected vegetables, such as e.g. peels from potato peels and cutting residuals from French fries production, pea cream, refused potatoes from trading, and also palm mill residues including palm oil mill effluent (POME) containing predominantly palm oil and palm oil fatty acids and empty fruit bunches (EFB) or palm fronds. Preferred feedstocks can be stored in silage, so it can be processed into SCP year round, while the feedstock is harvested in a campaign such as in the case of sugar beet pulp or the leaves of potato or sugar beet. Also, silages from whole fodder beet can be used, e.g. combined with corn or whole corn or the ensilaged form of thereof, although the lignin rich corn stover is not preferred, neither sugar cane bagasse. Pentoses e.g. from lignocellulosic hydrolysates can also be used. These syrups contain mainly glucose, xylose, arabinose, mannose and galactose. Another suitable source of raw material as fermentable carbon-rich feedstock for the processes of the invention is the organic fraction of municipal solid waste (MSW).

The method according to the invention is very useful for the production of pasteurised SCP that can be used in food or feed products. For the production of SCP for the manufacture of food products (for human consumption), any product of plant origin that is compatible with or acceptable for application in food can be applied in the invention as carbon-rich feedstock to use in provision of a biomass for pasteurisation, including e.g. corn, potato, wheat, rice, cassava, sugar cane or sugar cane juice, sugar beet or sugar beet juice or thick juice, molasses, cane molasses, glucose syrups, fructose syrups, of any other vegetable product suitable for food application. A lipid rich fraction, e.g. vegetable oils or fractions therefrom, can also be applied in the invention as carbon-rich feedstock, as the selected organisms also consume triglycerides, including e.g. soybean oil, olive oil, corn oil, palm oil, coconut oil, rapeseed oil, or sunflower oil etc.

The medium used for provision of biomass further preferably contains and/or is fed with a source of nitrogen. Preferably, the nitrogen source comprises one or more of (a source of) ammonia, urea and nitrate. More preferably, as a nitrogen source are the reduced form such as urea and ammonium. NH₃ or HNO₃ can additionally be to control pH in the fermenter or urea can be used as a pH-independent supply of nitrogen source. Also preferred are nitrogen sources from waste streams. These include e.g. one or more of amines present in burden condensates obtained from evaporation of molasses, sugar beet or cane vinasses, vinasses from wine industry, grape residues, potato protein liquor (PPL), Corn steep liquor (CSL), ammonia from animal farm exhaust gas cleaning scrubbers, and the thin fraction of manure processing.

In preferred embodiments of a method according to the invention, the biomass is grown or cultured in a chemically defined medium. The term “chemically defined” is understood to be used for fermentation media which are essentially composed of chemically defined constituents, i.e. the chemical composition of essentially all the chemicals used in the media is known. A fermentation medium which is essentially composed of chemically defined constituents includes a medium which does not contain a complex carbon and/or nitrogen source, i.e. which does of contain complex raw materials having a chemically undefined composition. The chemically defined media preferably do not comprise chemically ill-defined yeast, animal, or plant tissues; they do not comprise peptones, extracts, or digests or other components which may contribute chemically poorly defined proteins and/or peptides and/or hydrolysates. Chemically undefined or poorly defined chemical components are those whose chemical composition and structure is not well known, are present in poorly defined and varying composition, or could only be defined with enormous experimental effort.

Nonetheless, a fermentation medium which is essentially composed of chemically defined constituents may however further include a medium which comprises an essentially small amount of a complex nitrogen and/or carbon source, an amount as defined below, which typically is not sufficient to maintain growth of the microorganism and/or to guarantee formation of a sufficient amount of biomass. In that regard, complex raw materials have a chemically undefined composition due to the fact that, for instance, these raw materials contain many different compounds, among which complex heteropolymeric compounds, and have a variable composition due to seasonal variation and differences in geographical origin. Typical examples of complex raw materials functioning as a complex carbon and/or nitrogen source in fermentation are soybean meal, cotton seed meal, corn steep liquor, yeast extract, casein hydrolysate, molasses, et cetera.

An essentially small amount of a complex carbon and/or nitrogen source may be present in the chemically defined medium of the invention, e.g. as carry-over from the inoculum for the main fermentation. The inoculum for the main fermentation is not necessarily obtained by fermentation on a chemically defined medium. Most often, carry-over from the inoculum will be detectable through the presence of a small amount of a complex nitrogen source in the chemically defined medium for the main fermentation. It can be advantageous to use a complex carbon and/or nitrogen source in the fermentation process of the inoculum for the main fermentation, for instance to speed up the formation of biomass, i.e. to increase the growth rate of the microorganism, and/or to facilitate internal pH control. For the same reason, it may be advantageous to add an essentially small amount of a complex carbon and/or nitrogen source, e.g. yeast extract, to the initial stage of the main fermentation, especially to speed up biomass formation in the early stage of the fermentation process. An essentially small amount of a complex carbon and/or nitrogen source which may be present in the chemically defined medium of the invention is herein defined to be an amount of at the most about 10% of the total amount of carbon and/or nitrogen (Kjeldahl N) which is present in the chemically defined medium, preferably an amount of at the most 5, 2, 1, 0.5, 0.2 or 0.1% (w/v) of the total amount of carbon and/or nitrogen.

In a preferred embodiment, however, no complex carbon and/or nitrogen source is present in a chemically defined medium of the invention, other than an optional defoaming agent in as far as the defoaming agent can be used as a carbon source by the biomass strain such as a thermophilic fungus as cultured in a process of the invention. Fungal strains are preferably grown on a relatively pure carbohydrate (e.g. glucose) solution and ammonia in combination with a mineral salts solution (e.g. as described in US20140342396A1). The pure carbohydrate/sugar solutions allow the production with low levels of heavy metals, low toxic substances like herbicides, pesticides and fungicides, as well as low levels of mycotoxins derived from the feedstock, that may have been moulded during growth on the land, and or during storage and processing.

It is further to be understood that the term “chemically defined medium” as used herein, includes a medium wherein all necessary components are added to the medium before the start of the fermentation process, and further includes a medium wherein at least a part of the necessary components are added before starting and part are added or fed to the medium during the fermentation process.

A chemically defined medium to be used in the process of the invention typically contains so-called structural as well as so-called catalytic elements. Structural elements are understood as those elements which are constituents of microbial macromolecules, i.e. hydrogen, oxygen, carbon, nitrogen, phosphorus and sulphur. The structural elements hydrogen, oxygen, carbon and nitrogen typically are contained within the carbon and nitrogen sources. Phosphorus and sulphur typically are added as phosphate and sulphate and/or thiosulphate ions. The type of carbon and nitrogen source which is used in the chemically defined medium is not critical to the invention, provided that the carbon and nitrogen source have essentially a chemically defined character.

In a preferred embodiment, the carbon source in the chemically defined medium is or consists of a hydrophilic carbon source such as e.g. a carbohydrate. The inventors have found that the problem with fungal pellet morphology occurs when the fungus is grown at low pH in a chemically defined medium that lacks hydrophobic substances such as lipids (e.g. because the carbon source is hydrophilic) and that a desired dispersed morphology can be induced by including a small amount of hydrophobic substance, i.e. defoaming agent, in the medium. Thus the process preferably comprises growing a microorganism such as a strain of a thermophilic fungus in a chemically defined medium, at a pH of less than 5.0 wherein the chemically defined medium comprises at least one hydrophobic compound or substance. Preferably the hydrophobic compound or substance is a defoaming agent. Defoaming agents are generally well-known in the art (see e.g. en.wikipedia.org/wiki/Defoamer). A defoaming agent is a chemical additive that reduces and hinders the formation of foam in industrial process liquids, such as fermentation broths. While strictly speaking, defoamers eliminate existing foam and anti-foamers prevent the formation of further foam, the terms defoaming agent, anti-foam agent and defoamer are herein used interchangeably. A preferred defoaming agent for use in a process of the invention comprises or consist of a vegetable oil, preferably an edible vegetable oil. A preferred (edible) vegetable oil for use as defoaming agent in a process of the invention is an oil is selected from the group consisting of canola (rapeseed) oil, coconut oil, corn oil, cottonseed oil, olive oil, palm oil, palm kernel oil, linseed oil, peanut oil, safflower oil, soya bean oil, sunflower oil and high-oleic sunflower oil.

More specifically, in one embodiment, a strain of the thermophilic fungus is cultured in a chemically defined medium, consisting of a carbon source, a nitrogen source and further components necessary for growth of the fungus, wherein the carbon source in the chemically defined medium consist of at least one of a hydrophilic carbon source and the defoaming agent. The hydrophilic carbon source preferably comprises or consists of at least one of carbohydrate and organic acid. Preferably, the carbohydrate comprises a source of at least one of glucose, fructose, galactose, xylose, arabinose, rhamnose, fucose, galactose and mannose, of which glucose and fructose are preferred, and glucose is most preferred. Suitable carbohydrate carbon sources comprising a source of e.g. glucose and/or fructose include e.g. maltose, isomaltose, maltodextrins, starch, glucose syrups (e.g. corn syrups such as HCFS), inverted (cane or sugar beet) sucrose, a crude starch, a starch liquefact (e.g. by liquefying using alpha amylase such as Liquozyme (Novozymes) or Veretase (BASF), inulin, raffinose, melibiose and stachyose. Organic acids that can be comprised in the carbon source include lactic acid, acetic acid, galacturonic acid, glucuronic acid. The defoaming agent can be used as (at least part of) the carbon source, e.g. when the defoaming agent comprises an oil that can be utilised as carbon source by the fungal strain, such as a vegetable oil as mentioned above.

The nitrogen source in the chemically defined medium to be used in the processes of the invention preferably comprises or consists of at least one of urea, ammonia, nitrate, ammonium salts such as ammonium sulphate, ammonium phosphate and ammonium nitrate, and amino acids such as glutamate and lysine. More preferably, a nitrogen source is selected from the group consisting of ammonia, ammonium sulphate and ammonium phosphate. Most preferably, the nitrogen source is ammonia. The use of ammonia as a nitrogen source has the advantage that ammonia additionally can function as a pH-controlling agent. Preferably, when ammonia is used to control the pH, its concentration is controlled to be no more than 10, 20, 50, 100, 200, 500, 750 or 1000 mg/I. In case ammonium sulphate and/or ammonium phosphate are used as a nitrogen source, part or all of the sulphur- and/or phosphorus-requirements of the fungal strain may be met.

Catalytic elements are those elements which are constituents of enzymes or enzyme cofactors. These elements include e.g. magnesium, iron, copper, calcium, manganese, zinc, cobalt, molybdenum, selenium and borium. In addition to the aforementioned structural and catalytic elements, cations such as potassium and/or sodium preferably are present to function as a counter ion and for control of intracellular pH and osmolarity. Suitable mineral compositions for the chemically defined medium of the invention are described in US20140342396A1.

Compounds which may optionally be included in a chemically defined medium are buffering agents such as mono- and dipotassium phosphate, calcium carbonate, and the like. Buffering agents are preferably only added when dealing with processes without an external pH control.

Vitamins refer to a group of structurally unrelated organic compounds which may be necessary for the normal metabolism of thermophilic fungi. Fungi are known to vary widely in their ability or inability to synthesize the vitamins they require. A vitamin only needs to be added to the fermentation medium of a fungal strain incapable of synthesizing said vitamin. Typically, chemically defined fermentation media for lower fungi, e.g. Mucorales, may require supplementation with one or more vitamin(s). Higher fungi often have no vitamin requirement. Vitamins are selected from the group of thiamin, riboflavin, pyridoxal, nicotinic acid or nicotinamide, pantothenic acid, cyanocobalamin, folic acid, biotin, lipoic acid, purines, pyrimidines, inositol, choline and hemins.

In one embodiment, a thermophilic fungus that is grown in the process of the invention is a strain that does not require the presence of any vitamins in the chemically defined medium. The inventors have found that species of the genus Rhizomucor, e.g. the strains Rhizomucor pusillus CBS 143028, Rhizomucor miehei CBS 143029 and Rhizopus sp. CBS 143160, do not require any vitamins, even when grown on a mineral medium. In a preferred embodiment therefore, in step i) of the process the biomass is derived from a strain of thermophilic fungus that does not require any vitamins and that is grown in a chemically defined medium without added vitamins, preferably on a chemically defined medium consisting of a carbon source as herein defined above, a nitrogen source as herein defined above and minerals, e.g. as described in US20140342396A1. Further definitions for chemically defined medium are described in EP20153414.

The broth in which the biomass is fermented can have a reduced metal content, particularly when it is a chemically defined medium, which is preferred for food applications. Preferably, it has a reduced content of bivalent and trivalent metal salts, more preferably of bivalent metal salts, most preferably of iron salts. The metal ions from such salts generally accumulate in the cells of the biomass. Generally, when iron content is reported, it is reported as a fraction of dry matter. In preferred embodiments, the method is provided wherein the fermentation broth biomass comprises at most 1000 mg/kg dry matter iron, preferably at most 500 mg/kg, more preferably at most 350 mg/kg, even more preferably at most 150 mg/kg, still more preferably at most 75 mg/kg, most preferably at most 50 mg/kg. In other preferred embodiments, the iron content is at most 400, 300, 250, 200, 150, 140, 130, 120, 110, 100, 95, 90, 85, 80, 75, 70, 65, 60, 55, 50, 49, 48, 47, 46, 45, 44, 43, 42, 41, 40, 35, 30, 25, 20, 15, or 10 mg/kg. In other embodiments it is at most 100 mg/kg, preferably at most 50 mg/kg. Preferably, when the iron content is at most a given value, it is about that given value or within 50% below that value, more preferably within 25% or 10% below that value. Iron content can be determined using any method known in the art. Reduced iron content was surprisingly found to lead to a biomass composition with less colour, or a whiter colour, which can be desirable to facilitate later deliberate colouring.

A biomass can also be provided by other means. For instance a suitable biomass can be procured from a commercial supplier.

Step i) preferably also comprises a step i-b), which comprises processing of the fermentation broth of step i-a) to facilitate the subsequent pasteurisation of step ii). This can be via recovery or partial recovery of the biomass from fermentation, for example via concentration of the fermentation broth. In preferred embodiments, the fermentation broth such as that of step i-a) is concentrated to reduce energy demands of pasteurisation. This is preferably achieved via at least one of sieving, filtration and decantation. Preferably the biomass of step i-a) is sieved to obtain the provided biomass, more preferably using filtration such as via a sieving belt, a vibrating sieve, or a screen sieve.

The biomass can be concentrated by filtration, such as by at least one of rotating drum filtration, a filter press, a belt filter, a decanter centrifuge and sieving. Preferably biomass is recovered by sieving on a sieve or a screen, with 0.1, 0.5, 1 or 2 mm diameter of pores. More preferably, the biomass is recovered by at least two, three or four consecutive rounds of sieving on a sieve or screen whereby a smaller diameter of pores is applied in each subsequent round of sieving. For example a first round of sieving using 2 mm pore diameter, followed by subsequent rounds of 1, 0.5 and/or 0.1 mm. Preferably, the biomass is concentrated to a pumpable slurry, such as a pumpable slurry at >10% dry matter. This advantageously allows the pasteurisation to start on a smaller process volume. The pasteurisation of such a slurry can be done in a manner so that the equipment is small, allowing method operators to keep the required investments and operational costs low, and to minimize the energy needed to pasteurise the biomass. As described above, preferably, dry matter concentration of the sieved, filtered or decantated biomass is at least 7%, more preferably at least 10% when subjected to pasteurisation. In preferred embodiments it is about 7% to about 20%, or to about 15%. Most preferably it is about 9-12%. The microorganism such as the thermophilic fungus to be comprised in the provided biomass therefore preferably has good filtration properties.

In one embodiment of the process of the invention, the filtrate containing water and enzymes produced by the microorganism such as the fungus can be recycled and used in a next fermentation round. Preferably, water utilisation in the overall process is minimised. Preferably therefore in the process, the water fraction (filtrate) that is obtained after sieving, filtering, decanting and/or further pressing the biomass (cake) is recycled back to the fermentation and/or (re-)used for further fermentation batches. This is particularly preferred when the fermentation is run at low dry matter (e.g. less than 10, 5, or 2% dry matter). Preferably at least 10, 20, 50, 60, 70, 80, 90, 95, 96, 97, 98, 99, or more % of the filtrate from the recovery process is recycled. Preferably substantially all filtrate is recycled. If as a result of recycling salts and non-consumables accumulate in too high concentrations, part of the filtrate may be bled to the waste water treatment and/or used for fertilizer production. Preferably therefore, the titrants in the process are chosen such that a suitable fertilizer composition may be obtained from the filtrate, preferably a composition comprising one or more of N, P, K, S, Mg and Ca. Recycling of the water fraction will improve the overall economics of the process by reducing waste water treatment capacity and/or fresh water usage. Optionally, the filtrate of a first fermentation can be used in a second fermentation that allows a second organism to consume the carbon source that is not consumable by the first organism. Also application of two or more organisms in one fermentation would be possible. The application of two different thermophilic fungi, either simultaneously or subsequently in two or more fermentation runs, would allow optimization of yield, amino acid profile, taste, physical behaviour and many more. In a preferred embodiment, the filtrate obtained from a first fermentation with one or more strains of thermophilic fungi is used in a second fermentation with one or more strains of thermophilic fungi whereby at least one thermophilic fungal strain in the second fermentation differs from the strains used in the first fermentation. Preferably, the strains for the first and second fermentations are chosen to be complementary in terms of amino acid profiles of their biomass and/or capability to consume fractions of the carbon-rich feedstock. Preferably, the strains of thermophilic fungi that are used in the first and second, and optionally further fermentations, are selected from the thermophilic fungi and strains mentioned hereinabove. An example of two complementary thermophilic fungi that may be used subsequently are e.g. a strain of Thermomucor and a strain of Rasamsonia, or a strain of Thermomucor and a strain of Rhizomucor, or a strain of Rhizomucor and a strain of Rasamsonia.

Alternatively, enzymes can be recovered after filtration and sold as enzyme preparation for use in animal feed or detergent washing, industrial cleaning, etc.

Another advantage of the use of thermophilic fungi is that a fermenter can be operated without any cooling, or with minimal cooling depending on growth rate and biomass concentration (Suman et al., 2015 supra), e.g. without any (active) cooling device that requires an input of energy. Thus, neither an internal cooling coil in the fermenter nor cooling coil in baffles of a stirred fermenter, nor in fermenter wall, neither Riesel cooling is required, neither a cooling tower. An external cooling loop using a heat exchanger is not needed either. This will reduce the investment in the plant or setup as the cooling relies only on evaporation of water which will leave the fermenter via the gas exhaust of the fermenter via which the CO₂ is ventilated and/or heat that passively exchanged with the fermenter's environment.

Preferably, fermentation is performed in a fermenter that has a means for introducing sterile air (to prevent foreign fungal spores or yeasts to invade) and, preferably a means to control pH with e.g. NH₃ and/or H₂SO₄, HNO₃, or H₃PO₄ In some instances also a need for phosphate might be apparent and in such cases the use of ammonium phosphate or optionally H₃PO₄ is preferred in the processes of the invention. As is known to a skilled person, in some instances other minerals and trace elements can be preferred as well. The fermenter used for provision of biomass can be in principle be any type of fermenter known in the art. Advantageously the fermenter is a simple bubble column, which can be operated at very large scale such as e.g. >100 m³, >200 m³, >500 m³, >1000 m³, >2000 m³ or >3000 m³, thereby reducing the number of fermenters per factory, the total investment and operational cost.

### Method Step ii) Adding an Antioxidant

In step ii) an antioxidant (AOX) is added to the biomass to obtain a biomass mixture. Antioxidants are widely known, and a skilled person can select an antioxidant that is suitable for use in food or feed applications. In preferred embodiments, the antioxidant is at least one of:
a) a free radical scavenger, such as carnosol; carnosic acid; rosemary extract; a flavan-3-ol such as epicatechin, epigallocatechin, epigallocatechin gallate (EGCG), epicatechin gallate; a flavonoid such as kaemferol, quercetin, or myricetin; green tea extract; butylated antioxidants such as butylated hydroxytoluene (BHT), butylated hydroxyanisole, or tert-butylhydroquinone (TBHQ); gallic acid; a gallate ester such as propyl gallate; carvone; a caffeate ester such as methyl caffeate, ethyl caffeate, caffeic acid phenethyl ester (CAPE), or rosmarinic acid; spearmint extract; a vitamin E such as a tocopherol or a tocotrienol; a vitamin A such as retinol, retinoic acid, or retinal; a provitamin A such as a carotenoid, β-carotene, lutein, zeaxanthin, or echinenone; a quinolone-based antioxidant such as ethoxyquin; or seaweed extract; or
b) an oxygen reducer, such as ascorbic acid or acerola extract; or
c) a chelator, such as citric acid or ethylenediaminetetraacetic acid (EDTA), preferably disodium EDTA.

Other suitable antioxidants are E-numbers that are indicated to be antioxidants. More preferably, the antioxidant is a free radical scavenger. Preferred free radical scavengers are carnosol; carnosic acid; rosemary extract; a flavan-3-ol such as epicatechin, epigallocatechin, epigallocatechin gallate (EGCG), epicatechin gallate; a flavonoid such as kaemferol, quercetin, or myricetin; green tea extract; a caffeate ester such as methyl caffeate, ethyl caffeate, caffeic acid phenethyl ester (CAPE), or rosmarinic acid.

Mixtures of antioxidants can be used. For instance, rosemary extract is a mixture that comprises carnosol and carnosic acid. Green tea extract is a mixture that comprises flavan-3-ols such as epigallocatechin and epigallocatechin gallate, and flavonoids. Preferred mixtures of antioxidants comprise carnosol. Other preferred mixtures of antioxidants comprise a flavan-3-ol, preferably epigallocatechin gallate. Mixtures of antioxidants can comprise further excipients such as E433 (polyoxyethylene sorbitan monooleate), E1520 (propylene glycol), E471 (mono- and diglycerides of edible fatty acids), and E322 (Lecithin). Such mixtures are commercially available, for example under the NaturFORT brand from Kemin Food Technologies EMEA (Herentals, Belgium). A preferred mixture of antioxidants comprises rosemary extract and green tea extract. In some embodiments this mixture further comprises E1520, E471, and E322, for an oil soluble mixture. In other embodiments this mixture further comprises E433 (polyoxyethylene sorbitan monooleate), E1520, and E471, for a water soluble mixture.

Antioxidants can be water soluble or oil soluble or both. Preferred antioxidants are at least oil soluble, more preferably they are predominantly oil soluble. Oil soluble antioxidants can accumulate inside the cells of the biomass which promotes their retention during washing. A skilled person will appreciate that in this context solubility relates to effective solubility, which in mixtures can depend on excipients. When the antioxidant is in a mixture, the same antioxidant compound per se can be considered water soluble or oil soluble depending on any further excipients.

In eventual food or feed products, antioxidants are generally present at relatively low amounts. Preferably, at most about 5000 ppm of antioxidant is added to the biomass to obtain the biomass mixture. More preferably at most about 4000 or 3000 ppm is added. In preferred embodiments, at most about 2000 ppm is added, more preferably at most 1500 ppm, even more preferably at most 1000 ppm is added. Also preferred is the addition of at most 900, 800, 700, 600, 500, 400, 300, 200, or 100 ppm. Generally, it is preferred to add at least 50 ppm, more preferably at least 100, 200, 300, 400, 500,600, 700, 800, 900, or 1000 ppm. In preferred embodiments at least 500 ppm is added, more preferably at least about 750 ppm such as 1000 ppm.

Antioxidant can be added to the mixture in a single batch, or as a single step in the process. Alternately, antioxidants can be added spread over multiple doses such as 2, 3, or 4 doses. Preferably, antioxidant is added after step i) and before step iii) and before step iv). In preferred embodiments, antioxidant is only added during step ii). A skilled person will understand that this pertains to the deliberate addition of compounds that primarily act as antioxidants, and that other addition of matter to the composition, such as by dilution, is not intended to be referenced. It was found that the lowest TOTOX values were achieved when antioxidants were added at a single point during the method according to the invention, and that this was before any washing or pasteurisation had taken place.

In highly preferred embodiments, the antioxidant is added after step i) wherein step i) comprised concentrating the provided biomass, preferably via sieving, and before step iii) wherein the mixture is pasteurised, wherein step iv) comprises washing of the pasteurised mixture. It is highly preferred that the biomass is not washed at any other point during the process, more preferably it is not washed in between the moment where it is derived from a fermentation broth and the washing that is part of step iv).

In some embodiments, the antioxidant is added in two batches, with about 50% of the total intended amount being added after step i) wherein step i) comprised concentrating the provided biomass, preferably via sieving, and before step iii) wherein the mixture is pasteurised, wherein step iv) comprises washing of the pasteurised mixture, after which the remainder of the total intended amount of antioxidant is added. It is highly preferred that the biomass is not washed at any other point during the process, more preferably it is not washed in between the moment where it is derived from a fermentation broth and the washing that is part of step iv).

In some embodiments, the antioxidant is added after step i) wherein step i) comprised concentrating the provided biomass, preferably via sieving, and after step iii) wherein the mixture is pasteurised, and after step iv) wherein step iv) comprises washing of the pasteurised mixture.

In some embodiments, the antioxidant is added after step i) wherein step i) comprised concentrating the provided biomass, preferably via sieving, after which the concentrated biomass is washed, and before step iii) wherein the mixture is pasteurised.

Method Step iii) Pasteurisation of the Biomass

In step iii) the biomass is pasteurised to obtain pasteurised biomass. Pasteurisation is a technique that is well-known in the art, and can be performed using commercially available equipment. Pasteurisation can be done in a batch process, and it can also be done in a continuous process. Continuous process pasteurisation can be referred to as in-line pasteurisation, and is preferred for the method according to the invention because it reduces the complexity of the setup. When pasteurisation is done in a continuous process the method is a continuous method. In such cases the biomass provided in step i) can be described as a stream of biomass, and the pasteurised biomass can be referred to as a stream of pasteurised biomass.

Accordingly, in preferred embodiments, in step iii) the biomass is streamed through an in-line heating unit to obtain pasteurised biomass. In-line heating units are well known and various types are commercially available, several of which are actively marketed as in-line pasteurisation units or as flow pasteurisers. Such heating units can generally be set to a predefined temperature, and the residence time of a liquid flowing through the heating unit can generally be controlled. In principle any of these in-line heating unit can be used to practice the invention as long as the required residence time and temperature can be reached. In preferred embodiments, the duration of pasteurisation is expressed as the duration of contact with a heating unit. In preferred embodiments, the duration of pasteurisation is expressed as the duration of residence in an in-line heating unit. Preferably, residence time in an in-line heating unit is expressed as the amount of time the biomass is approximately at or above the indicated pasteurisation temperature. In preferred embodiments, the duration of pasteurisation is expressed as the time during which the biomass is approximately at or above the indicated pasteurisation temperature.

Although any heating unit known in the art can be used, it is preferred that the heating unit is an in-line heating unit such as a pipe heater or a heating block. In such an in-line heater liquid, in this case the biomass, flows through a channel in which it is heated to the desired temperature. Biomass enters the in-line heater through a first opening, is heated in the interior of the first channel, and leaves the first channel through the second opening. The heating is preferably controlled by one or more temperature sensors located in the heating unit, or just downstream thereof, preferably in a channel that transports pasteurised biomass. Preferably, two temperature sensors are located in the element. In a most preferred configuration, one sensor is located near or at the inflow opening and one sensor is located near or at the outflow opening.

In preferred embodiments is provided the method according to the invention, wherein the pasteurisation is performed in an in-line heating unit that preferably comprises a pipe heater, a heating block, or a steam infusion element, more preferably a steam infusion element, and wherein the in-line heating unit optionally comprises a mixing element such as a static mixer.

For a pipe heater, the in-line heating unit is configured as a pipe, typically an aluminium or stainless steel pipe, which is wound with a heating wire, or fitted with a jacket that is heated with hot water or steam. In such cases the interior of the pipe is the channel through which biomass is transported. The heating wire is in intimate contact with this channel. A preferred heating unit comprises a heating wire enclosed inside an aluminium tube filled with electrically non-conductive medium, such as magnesium oxide powder. For a heating block, the in-line heating unit is configured as a block, typically a thermally conductive block, which is traversed by the biomass channel, and which heats up in its entirety. This can be via an integrated heating wire, via a traversing heating wire, or via other means known in the art. Suitable in-line heating units are preferably formed from thermally conductive materials. Suitable materials are ceramics and metallic materials such as stainless steel, cast iron, copper, aluminium, brass, zinc, and alloys thereof.

A steam infusion element is preferred. Steam infusion is widely known (Encyclopedia of Agricultural, Food, and Biological Engineering, Second Edition. pp. 1581-1586, doi:10.1081/E-EAFE2-120045618). A benefit of heating with direct steam is that the design is simpler, there is no condensate returning, and it will reduce fouling on the wall. Normally when heating via the wall there will be a temperature gradient from the wall to the core of the pipe. As a result the biomass could bake to the wall. This is avoided by steam injection. Preferably, when steam injection is used, it is used in combination with a biomass comprising at least 8%, preferably at least 10% dry matter. Steam preferably consists essentially of water without further additives; for instance, preferred steam is food grade steam or culinary steam.

As is known in the art, for in-line heating units the combination of flow speed, internal channel volume, and heating will determine the effective heating of the substance that is being heated. The channel through which the biomass flows when it is to be pasteurised in the in-line heating unit therefore preferably has a diameter that is configured to allow a certain residence time inside the heating unit. Preferably, the diameter is at most 30, 25, 20, 15, 14, 13, 12, 11, 10, 9, 8, 7, 6, 5, 4, 3, 2, or 1 cm. Preferably the diameter is at least 0.5, 1, 2, 3, 4, 5, 6, 7, 8, 9, or 10 cm. Preferably the flow speed of the biomass is at least about 0.05 m/s, more preferably at least 0.1 m/s, even more preferably at least 0.25 m/s. In some embodiments the flow speed is at most 2 m/s.

Heating is conveniently expressed as the amount of time that a liquid has resided at any given temperature. Preferably the indicated time does not include the time required for a liquid to equilibrate or to approximately equilibrate with the prevailing temperature in the heating unit. For convenience, the temperature of the biomass at pasteurisation conditions is referred to as the temperature of the heating unit. For a batch process, the time is generally the same as the duration of the process. Fora continuous process, for example using an in-line heating unit, the duration is generally expressed as a residence time of the liquid inside the heating unit. When an in-line heating unit is longer, the same residence time can be achieved at higher flow. This reduces baking of the biomass onto the wall of the channel.

An in-line heating unit optionally comprises a mixing element such as a static mixer. Such measures are well known in the art (Paul, Edward L. (2004). Handbook of Industrial Mixing-Science and Practice. Hoboken N.J.: John Wiley & Sons. pp. 399 section 7-3.1.4), and are beneficial when a pipe has a larger diameter because they ensure a more even distribution of heat. For more narrow pipes, such as pipes having a diameter of at most 5 cm, it is preferred that no static mixers are present. Preferred static mixers are plate-type static mixers, helical static mixers, and baffles.

In step iii), the biomass has a residence time in the heating unit of at most 45 minutes at a temperature of at least 70° C. Preferably, when a temperature is indicated to be at least a given temperature, the temperature at which the pasteurisation is performed is close to that given temperature, preferably it is not more than 20, more preferably not more than 10, even more preferably not more than 5° C. above the indicated temperature. This helps reduce energy demand. When heating is indicated to be for at most a given amount of time, it is preferably performed for about the indicated amount of time. This contributes to the uniformity of the processes. Pasteurisation for more than the indicated amount of time leads to increased energy consumption and can contribute to the development of off-flavours in the biomass. Pasteurisation for substantially shorter than the indicated amount of time can lead to insufficient reduction of germ count. The inventors found that these parameters led to adequate reduction of germ count in the biomass. In preferred embodiments, pasteurisation is performed for 37 minutes at 74° C., which was found to lead to a spore germ count log₁₀ reduction of 12.

In the context of this application, one of the main goals of pasteurisation is the reduction of germ count in the biomass. Germ count can be based on mycelium count. Germ count can also be based on spore count. Preferably in the context of this application the reduction encompasses spore count in addition to mycelium count. Reduction of germ count is conveniently expressed as a log₁₀ reduction, where a log₁₀ reduction of 2 means that only about 1 out of 100 microbes survive the treatment, and a log₁₀ reduction of 7 means that only about 1 out of 10000000 microbes survive. This is a common format for reporting such data. Log₁₀ reduction can be assessed using any methods known in the art, such as dye reduction tests using for example methylene blue or resazurin (see for example Bapat et al., J Microbiol Methods. 2006 April; 65(1):107-16, DOI: 10.1016/j.mimet.2005.06.010). More preferably dilution series are plated and colony forming units are counted. Heat inactivation kinetics are preferably determined as described by Casolari (2018, Microbial death, Physiological Models in Microbiology: Volume II). Preferably, the germ count of the pasteurised biomass has a log₁₀ reduction, as compared to the provided biomass, of at least 7, as this substantially prolongs shelf life of pasteurised biomass. A typical food norm for fungi is that a germ count should be below 10 fungi per gram of food. Of course this depends on the amount of single-cell protein that is present in the food, as well as on the germ count of other ingredients that can be present in the food.

In preferred embodiments is provided the method according to the invention, wherein the germ count of the pasteurised biomass has a log₁₀ reduction of at least 7, preferably at least 8, more preferably at least 9, still more preferably at least 10, more preferably still at least 11, most preferably of at least 12, as compared to the provided biomass. Accordingly, step iii) is preferably performed at such temperature, or for such a duration, so as to achieve this log₁₀ reduction. Based on the examples and the teachings provided herein a skilled person can adjust these parameters to achieve this reduction.

Another goal of pasteurisation is the reduction of enzymatic activity in the biomass. Enzymes can contribute to degradation of cellular components, and degradation products can contribute to the development of an off-taste in the SCP. Preferably, the method according to the invention reduces the activity of one or more enzymes that catalyse or promote the oxidation and/or degradation of at least one of (poly)peptides and lipids, more preferably of at least one of a lipase, lipoxidase, peptidase, protease, and an aminopeptidase. These enzymes are widely known. Examples of peptidases are endopeptidases and exopeptidases. More preferably, the activity of two, most preferably of each of these enzymes is reduced. Reduction of activity in pasteurised biomass is to be compared to activity in unpasteurised biomass. Reduction of total activity for the enzymes to be assayed is preferably at least 1, 5, 10, 20, 30, 40, 50, 60, 70, 80, 90, 95, 98, 99, or 100%, which can mean that no remaining activity is detectable. Reduction is more preferably at least 10%, even more preferably at least 80%. A skilled person knows how to assess the reduction of enzymatic activity, and can select a suitable assay for any of the described enzyme types.

A shorter duration of the pasteurisation or of the exposure to heat or of the residence in the heating unit can lead to improved efficiency, because it allows a larger volume of biomass to be processed in a shorter amount of time. For batch processes, it would allow more processes to be performed in a given time frame. Also, short heating generally reduces energy consumption. In preferred embodiments, the pasteurisation is performed for at most 40, 35, 30, 29, 28, 27, 26, 25, 24, 23, 22, 21, 20, 19, 18, 17, 16, 15, 14, 13, 12, 11, 10, 9, 8, 7, 6, 5, 4.5, 4, 3.5, 3, 2.5, 2, 1.5, 1, 0.9, 0.8, 0.7, 0.6, 0.5, 0.4, 0.3, 0.2, or 0.1 minutes. More preferably it is performed for at most 30 minutes. Still more preferably for at most 25 minutes. Still more preferably for at most 20 minutes. Still more preferably for at most 15 minutes. Still more preferably for at most 10 minutes. Still more preferably for at most 5 minutes. Still more preferably for at most 3 minutes. Still more preferably for at most 2 minutes. Most preferably for at most 1 minute. In preferred embodiments, the pasteurisation is performed for at most 5 minutes, preferably from about 0.5 to about 3 minutes, more preferably from about 1 to 2 minutes.

A minimum amount of pasteurisation duration can help ensure adequate germ count reduction. In preferred embodiments the pasteurisation is performed for at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, or 10 minutes. More preferably it is performed for at least 1 minute. In other preferred embodiments it is performed for at least 2 minutes. In other preferred embodiments it is performed for at least 3 minutes. In other preferred embodiments it is performed for at least 4 minutes. In other preferred embodiments it is performed for at least 5 minutes. In other preferred embodiments it is performed for at least 6 minutes.

Pasteurisation at higher temperature can help achieve the same germ count reduction during a shorter time duration of pasteurisation. In preferred embodiments pasteurisation is performed at a temperature of at least 74° C., preferably at least 80° C., more preferably at least 86° C. In other preferred embodiments, pasteurisation is performed at a temperature of at least 70, 71, 72, 73, 74, 75, 76, 77, 78, 79, 80, 81, 82, 83, 84, 85, 86, 87, 88, 89, 90, 91, or 92° C. More preferably it is performed at at least 75° C., still more preferably at at least 77° C., still more preferably at at least 79° C., more preferably still at at least 81° C., still more preferably at least 82° C., even more preferably at least 83° C., even more preferably at least 84° C., where 85° C. is even more preferred, and 86° C. even more. Pasteurisation at 87° C. even allows log₁₀ reduction of 7 in under half a minute. Pasteurisation at 88° C. even allows log₁₀ reduction of 12 in about half a minute.

In some embodiments, the pasteurisation in step iii) is performed twice. In preferred embodiments it is performed once.

Pasteurisation is preferably performed at a pH below 5, more preferably below 4.5, even more preferably below 4, 4.9, 4.8, 3.7, 3.6, 3.5, 3.4, 3.3, 3.2, 3.1, or 3, most preferably at about 3.5. Pasteurisation is preferably performed at a pH of at least 1, more preferably at least 2, even more preferably at least 2.5, 2.6, 2.7, 2.8, 2.9, or 3, most preferably at least about 3.5. pH of the biomass to be pasteurised can be controlled as described for step i), as the pH is generally that of the provided biomass. Preferably, the pH is not adjusted between step i) and step iii). Preferably the pH is not adjusted as part of step ii) or iii), more preferably as part of neither. pH during pasteurisation is preferably substantially similar to the pH at which fermentation is performed when fermentation is performed as part of step i), or to the pH at which the fermentation was from which the biomass was derived in step i). In preferred embodiments, the pH of the biomass in step iii) is at most 4.5, preferably at most 3.5. In preferred embodiments the pH of the biomass composition in step iv) is at most 4.5, preferably at most 3.5.

When pasteurisation is performed for at most 45 minutes, it is preferably performed at at least 72° C. When pasteurisation is performed for at most 40 minutes, it is preferably performed at at least 73° C. When pasteurisation is performed for at most 35 minutes, it is preferably performed at at least 73° C. When pasteurisation is performed for at most 30 minutes, it is preferably performed at at least 75° C. When pasteurisation is performed for at most 25 minutes, it is preferably performed at at least 74° C. When pasteurisation is performed for at most 20 minutes, it is preferably performed at at least 75° C. When pasteurisation is performed for at most 15 minutes, it is preferably performed at at least 77° C. When pasteurisation is performed for at most 10 minutes, it is preferably performed at at least 77° C. for a log₁₀ reduction of 8, or at 78° C. When pasteurisation is performed for at most 9 minutes, it is preferably performed at at least 77° C. When pasteurisation is performed for at most 8 minutes, it is preferably performed at at least 78° C. When pasteurisation is performed for at most 7 minutes, it is preferably performed at at least 79° C. When pasteurisation is performed for at most 6 minutes, it is preferably performed at at least 80° C. When pasteurisation is performed for at most 5 minutes, it is preferably performed at at least 79° C. When pasteurisation is performed for at most 4 minutes, it is preferably performed at at least 81° C. When pasteurisation is performed for at most 3 minutes, it is preferably performed at at least 81° C. for a log₁₀ reduction of 7, or at 83° C. for a log₁₀ reduction of 12. When pasteurisation is performed for at most 2 minutes, it is preferably performed at at least 82° C. for a log₁₀ reduction of 7, or at 84° C. for a log₁₀ reduction of 12. When pasteurisation is performed for at most 1 minute, it is preferably performed at at least 84° C. for a log₁₀ reduction of 7, or at 86° C. for a log₁₀ reduction of 12.

When pasteurisation is performed at at least 72° C., it is preferably performed for at most 45 minutes. When pasteurisation is performed at at least 74° C., it is preferably performed for at most 40 minutes. When pasteurisation is performed at at least 76° C., it is preferably performed for at most 12 minutes (log₁₀ reduction of 7) or for at most 20 minutes (log₁₀ reduction of 12). When pasteurisation is performed at at least 78° C., it is preferably performed for at most 7 minutes (log₁₀ reduction of 7) or for at most 11 minutes (log₁₀ reduction of 12). When pasteurisation is performed at at least 79° C., it is preferably performed for at most 5 minutes (log₁₀ reduction of 7) or for at most 8 minutes (log₁₀ reduction of 12). When pasteurisation is performed at at least 80° C., it is preferably performed for at most 4 minutes (log₁₀ reduction of 7) or for at most 6 minutes (log₁₀ reduction of 12). When pasteurisation is performed at at least 81° C., it is preferably performed for at most 2.5 minutes (log₁₀ reduction of 7) or for at most 4.5 minutes (log₁₀ reduction of 12). When pasteurisation is performed at at least 82° C., it is preferably performed for at most 2 minutes (log₁₀ reduction of 7) or for at most 3.5 minutes (log₁₀ reduction of 12). When pasteurisation is performed at at least 83° C., it is preferably performed for at most 1.5 minutes (log₁₀ reduction of 7) or for at most 2.5 minutes (log₁₀ reduction of 12). When pasteurisation is performed at at least 84° C., it is preferably performed for at most 1 minute (log₁₀ reduction of 7) or for at most 2 minutes (log₁₀ reduction of 12). When pasteurisation is performed at at least 85° C., it is preferably performed for at most 1.5 minutes (log₁₀ reduction of 12). When pasteurisation is performed at at least 86° C., it is preferably performed for at most 1 minute (log₁₀ reduction of 12).

Pasteurisation at at least 78° C. was found to be associated with improved organoleptic qualities. Temperatures of 80° C. are preferred in this context, with 84° C. allowing log₁₀ reduction of 7 during just one minute. Excellent results were obtained at 86° C., it is preferably performed for at most 12 minutes (log₁₀ reduction of 7) or for at most 20 minutes (log₁₀ reduction of 12).

Pasteurisation for at most 12 minutes was found to be associated with improved organoleptic qualities. A duration of at most 6 minutes is preferred in this context, with at most 4.5 minutes allowing log₁₀ reduction of 12 at 81° C. Excellent results were obtained with pasteurisation of at most about 3 minutes. In this context, it is preferably performed for at most 2 or 1 minutes.

After pasteurisation, the obtained biomass composition preferably has a TOTOX value under 200, more preferably under 150, even more preferably under 150, still more preferably under 100. In preferred embodiments, the method according to the invention is provided, wherein the obtained biomass composition has a TOTOX value of under 50, preferably under 35, more preferably under 20, and wherein the TOTOX value preferably remains under 50, more preferably under 40, for at least 6 weeks, more preferably for at least 1 year.

As described elsewhere herein, the TOTOX value is the sum of the anisidine value (AV) plus twice the peroxide value (PV). Methods for determining PV and AV are known in the art. Preferably, AV is determined following NEN-EN-ISO 6885:2000. PV is the milliequivalents of peroxide oxygen combined in a kilogram of material and is preferably determined by having it liberate iodine from potassium iodide, which is then estimated using a standard sodium-thiosulfate solution (Methods of Analysis of Oils and Fats, 1958; Official and Tentative Methods of the American Oil Chemists' Society).

Primary oxidation processes in oil mainly form hydroperoxides, which are represented by the PV. As secondary oxidation products appear, PV does not increase further but increases. PV is a good way to represent the amount of primary oxidation products in fresh SCP. SCP with significant levels of peroxides may still be odourless if secondary oxidation has not begun. If oxidation is more advanced, PV may be low but the SCP will obviously have inferior smell such as a rancid smell. A lower PV is preferred. A PV below 10 is preferred, below 6 is more preferred, even more preferably it is below 3. The secondary stage of oxidation occurs when hydroperoxides decompose to form carbonyls and other compounds, in particular aldehydes. Such aldehydes are represented by the AV, and can lead to a rancid smell in SCP. A lower AV is preferred. Preferably AV is below 30, more preferably below 10.

The TOTOX value preferably remains below a given value for a prolonged period of time. Preferably, it remains below a given value for at least 4 weeks, more preferably for at least 6 weeks, even more preferably for at least 6 months, most preferably for at least a year. When products such as SCP are stored at elevated temperatures such as at 55° C. this can be taken as a model for storage of about 1.3 years at 20° C. As is known in the art, as a model for accelerated shelf life assessment, an increase of 10° C. in storage temperature can represent a doubling of the storage duration. For instance, storage for 6 weeks at 30° C. can serve as a model for storage of 12 weeks at 20° C. In preferred embodiments, the TOTOX value remains below a given value for at least about a year.

After at least one year at 20° C. the TOTOX value is preferably below 50, more preferably below 45, even more preferably below 35, still more preferably below 25. After at least six weeks at 55° C. the TOTOX value is preferably below 50, more preferably below 45, even more preferably below 35, still more preferably below 25. After at least one year at 20° C. the PV is preferably below 20, more preferably below 15, even more preferably below 8. After at least six weeks at 55° C. the PV is preferably below 20, more preferably below 15, even more preferably below 8. After at least one year at 20° C. the AV is preferably below 20, more preferably below 10, even more preferably below 6, still more preferably below 5 or 4. After at least six weeks at 55° C. the AV is preferably below 20, more preferably below 10, even more preferably below 6, still more preferably below 5 or 4.

Method Step iv) Washing and/or Recovery of the Pasteurised Biomass Composition

Step iv) is an optional step, and can comprise washing of the biomass composition. Preferably, the washing of step iv) is the only washing step that is performed during the method according to the invention. Washing can help isolate the SCP by removing fermentation broth. Washing can change the pH of the composition. Washing is performed using a suitable liquid washing medium, preferably water, more preferably water fit for feed or food applications, such as tap water or reverse osmosis purified water. Washing is conveniently performed using a filter or sieve or screen that substantially retains all solids. Washing can also be performed by serial dilution and decanting, filtering, or centrifugation. A preferred method for washing is counter current washing, which reduces required washing volumes. A skilled person can select a suitable washing technique. Preferably, washing is performed until the pH of the composition is in the range of 3.5-9, more preferably 4-8, even more preferably 4.5-7, most preferably until it is about 5-6. After washing, the pH is preferably at least 5. After washing, the pH is preferably at most 6. Washing is preferably performed until the composition comprises at most 40% (w/w) of substances other than SCP and washing medium, more preferably at most 15%, even more preferably at most 10, 9, 8, 7, 6, 5, 4, 3, 2, or 1%. In some embodiments washing is performed until at most 0.5% (w/w) of substances other than SCP and washing medium remain, preferably at most 0.1%, more preferably at most 0.05%.

Step iv) can also comprise recovery of the pasteurised biomass. This generally leads to an isolated SCP that can be used in further applications, preferably in a food or feed product.

In preferred embodiments of the method according to the invention, step iv) is not optional and comprises washing. In preferred embodiments of the method according to the invention, step iv) is not optional and comprises recovery of the biomass composition. In more preferred embodiments of the method according to the invention, step iv) is not optional and comprises both washing and recovery.

Methods for recovery of a pasteurised biomass can be the same as known methods for recovery of a biomass from a suspension. For example, the pasteurised biomass can be further concentrated as described in step i-b), but to a further extent, for instance to form a cake of pasteurised biomass. Preferably, dry matter concentration of the sieved, filtered or decanted biomass (cake) is at least 12%, 15%, 20%, 25%, 30%, 35%, 40%, 45%, 50%, 52%, 53% 54% or 55% (w/v). Optionally, dry matter concentration of the sieved, filtered or decanted biomass (cake) is further increased by further removal of water, i.e. drying.

When the used microorganism has good filtration properties (see above), a protein rich animal feed or food cake can be obtained by simple filtration such as rotating drum filtration, screen strap, filter press, belt filter etc. or a decanter centrifuge operated at low g forces (Suman et al., 2015 supra), a sieve, a DSM screen, belt sieve, belt press, screw press.

The recovered product can be moist. Recovered product can be stabilized by adding organic acids such as formic acid, acetic acid, benzoic acid, phosphoric acid, or sulphuric acid to prevent microbial deterioration, optionally combined, by keeping the pH≤4.5, preferably at or below 3.5. When pH of biomass provided in step i) is sufficiently low such as below 4.5, it can be kept in that range by for instance reducing the intensity of any dilution or washing of the recovered biomass. Although cost of production of liquid feeds and foods is generally lower, optional drying of the feed or food such as an animal feed cake using e.g. fluid bed drying, vacuum drying, drum drying, belt drying or any other means of drying can be considered if transport, logistics and/or storage stability demand this. In a particularly preferred process the concentration of the biomass is done in multiple steps and combinations: e.g. by subsequently sieving through pore sizes selected from at least two of 2 mm, 1 mm, 0.1 mm and 50 um; then concentrating by at least one of a screen strap, pressing using screw press, a hydraulic press and a pneumapress. In a most preferred process for concentrating the biomass can simply be the combination of a DSM screen (with optimized diameter screen), a screen strap and a belt press. The filtrate containing water and enzymes produced by the fungus can be recycled and used in a next fermentation round as described for step i-b).

The recovered pasteurised biomass can e.g. be further dried by pressing (more of) the residual water out using e.g. compressed air using a pneumapress and/or mechanical pressing, using e.g. a belt press or a screw press. In warmer climates the biomass (cake) can simply dried to the air (in the sun). After pressing the biomass to a cake, optionally the cake can be milled or extruded e.g. to enable drying, preferably air drying. Preferably, the particle size of the pressed mycelial biomass cake is reduced by physical means to enable (more efficient) drying of the pressed cake. This can optionally done by extrusion of the mycelial cake through holes with a diameter of 0.6, 0.7, 0.8, 0.9, 1.0, 1.2, 1.4, 1.6, 1.8 or 2 mm, using extruders that are known in the art. If however the dry matter concentration of the pressed cake after pressing is so high, that extrusion of the pressed cake is no longer possible (e.g. when the cake is too firm to allow for extrusion), the particle size of the cake can be reduced by a combination of milling and sieving. As a milling step any type of mill known in the art per se can be used, such as e.g. a knife mill or a hammer mill, etc. To obtain homogeneous particle size of the milled pressed cake, the larger particles still present after milling can be removed before drying by sieving with a pore diameter size in the sieve of 0.5, 1.0, 1.2, 1.4, 1.6, 1.8, 2.0, 2.5 or 3 mm. The resulting milled cake would have preferably a particle size between 1-3 mm before drying. By reducing the particle size, evaporation of water from the pressed cake is more efficient and faster.

Preferably drying of the cake is done by using waste heat, e.g. from a plant where hot water is obtained after condensation of gas (e.g. ethanol distillation, potato cooking, steam-pealing of potatoes, etc.). The air can be heated using a heat exchanger to heat up dry air with hot water from the heat source.

Drying of the extruded or milled cake is preferably done at temperatures of 30-70° C. The hot air can then dry the cake in a gentle and cost effective way in a belt dryer or fluid bed dryer. Steam drying at high temperatures (e.g. >80° C.) is preferably avoided as it can negatively influence digestibility of the proteins by denaturing and baking and even chemical decomposition of the amino acids by Maillard reactions. Flash drying under vacuum is another preferred method.

Preferably, when it is present, step iv) also encompasses a heat recovery technique. The heat that is generated by the pasteurisation and that remains in pasteurised biomass can conveniently be used to pre-heat the provided biomass that is yet to be pasteurised, or to heat biomass in a fermenter. For instance, the outlet channel and the inlet channel of the in-line heating unit can be configured in such a way as to allow heat exchange between the two channels. This can be done by leading the channels through a heat conductive medium such as through an aluminium block. Alternately the channels can be intertwined, or one can spiral around the other. A skilled person knows how to configure channels to allow heat exchange. Alternately, the outlet channel is configured in such a way as to allow heat exchange between the outlet channel and a fermenter, preferably a fermenter that ferments the biomass in step i). This allows the residual heat in the pasteurised biomass to contribute to maintaining the desired fermentation temperature. A convenient side effect of recovering heat from the pasteurised biomass is that it can help cool the pasteurised biomass.

Accordingly, in preferred embodiments, the method of the invention has a step iv) comprising recovering the pasteurised biomass and allowing heat exchange between the pasteurised biomass and an unpasteurised biomass, for example between the pasteurised stream of biomass and the stream of biomass provided in step i). Alternately, the heat can be exchanged between the pasteurised biomass and a fermenter from which the biomass provided in step i) is derived.

A biomass composition can also be freeze dried as part of step iv). Preferably, when it is freeze dried, it has been washed with water first.

### Further Definitions of the Pasteurisation Process and the Method

In preferred embodiments, in step i) a biomass derived from a fermentation, preferably having 2-5% dry matter, preferably a submerged fermentation, of a fungus, preferably a thermophilic fungus, is provided, which then preferably in step i-b) is sieved on a belt or vibrating sieve or DSM screen to achieve a dry matter content of 7-15% dry matter such as about 10% dry matter, and in step iii) the pasteurisation is preferably performed using an in-line heating unit, preferably comprising a steam injector.

In preferred embodiments, in step i) a biomass derived from a fermentation, preferably having 2-5% dry matter, preferably a submerged fermentation, of a fungus, preferably a thermophilic fungus, is provided, which then preferably in step i-b) is sieved on a belt or vibrating sieve or DSM screen to achieve a dry matter content of 7-15% dry matter such as about 10% dry matter, and in step iii) the biomass flows through an in-line heating unit, preferably comprising a steam injector, where it has a residence time of at most 10 minutes at a temperature of at least 76° C., preferably of at least 77° C.

In preferred embodiments, in step i) a biomass derived from a fermentation, preferably having 2-5% dry matter, preferably a submerged fermentation, of the genus Rhizomucor, preferably of the strain Rhizomucor pusillus, is provided, which then preferably in step i-b) is sieved on a belt or vibrating sieve or DSM screen to achieve a dry matter content of 7-15% dry matter such as about 10% dry matter, and in step iii) the biomass flows through an in-line heating unit, preferably comprising a steam injector, where it has a residence time at most 10 minutes, preferably of about 1 to 2 minutes, at a temperature of at least 77° C., preferably of at least 80° C., most preferably of about 86° C.

In preferred embodiments, in step i) a biomass derived from a submerged fermentation at 2-5% dry matter of the strain Rhizomucor pusillus is provided, which then preferably in step i-b) is sieved on a belt or vibrating sieve or DSM screen to achieve a dry matter content of 7-15% dry matter such as about 10% dry matter, and in step iii) the biomass flows through an in-line heating unit, preferably comprising a steam injector, where it has a residence time of about 1 to 2 minutes at a temperature of about 86° C.

In preferred embodiments, the provided biomass is derived from fermentation by sieving followed by addition of all antioxidant, which is then followed by pasteurisation, which is then followed by washing of the pasteurised composition. Preferably, steps of the method are performed in the order in which they are numbered. Preferably, a step occurs only once. Preferably, no other substantial steps or no other steps are comprised in the method.

In preferred embodiments, in step i) a biomass derived from a fermentation, preferably having 2-5% dry matter, preferably a submerged fermentation, of a fungus, preferably a thermophilic fungus, is provided, which then preferably in step i-b) is sieved on a belt or vibrating sieve or DSM screen to achieve a dry matter content of 7-15% dry matter such as about 10% dry matter, after which in step ii) at most 2500 ppm antioxidant is added, wherein the antioxidant is preferably a free radical scavenger, more preferably at least one of carnosol; carnosic acid; rosemary extract; a flavan-3-ol such as epicatechin, epigallocatechin, epigallocatechin gallate (EGCG), epicatechin gallate; a flavonoid such as kaemferol, quercetin, or myricetin; green tea extract, or a mixture comprising at least one thereof, more preferably one or both of rosemary extract and green tea extract, and in step iii) the pasteurisation is preferably performed using an in-line heating unit, preferably comprising a steam injector, after which in step iv) the composition is washed, wherein this wash is the only wash during the process, and wherein this addition of antioxidant is the only addition of antioxidant.

In preferred embodiments, in step i) a biomass derived from a fermentation, preferably having 2-5% dry matter, preferably a submerged fermentation, of a fungus, preferably of the strain Rhizomucor pusillus, is provided, which then preferably in step i-b) is sieved on a belt or vibrating sieve or DSM screen to achieve a dry matter content of 7-15% dry matter such as about 10% dry matter, after which in step ii) at most 2500 ppm antioxidant is added, preferably as defined in the paragraph above, and in step iii) the biomass flows through an in-line heating unit, preferably comprising a steam injector, where it has a residence time of about 1 to 2 minutes at a temperature of about 86° C., after which in step iv) the composition is washed, wherein this wash is the only wash during the process, and wherein this addition of antioxidant is the only addition of antioxidant.

### Products Obtained by the Method

The invention provides the biomass composition obtainable by the method according to the invention. It comprises an antioxidant and biomass, and preferably comprises from about 6% to 12% lipids and from about 35% to 55% proteins, based on dry weight. It is preferably pasteurised.

Lipid content of a biomass composition is preferably at least 4, 5, 6, 7, 8, 9, 10, 11, 12, 13, 14, or 15%. More preferably it is at least 6%, even more preferably at least 8%, more preferably still at least about 10%. Lipid content of a biomass composition is preferably at most 30, 28, 26, 24, 22, 20, 19, 18, 17, 16, 15, 14, 13, 12, 11, or 10%. More preferably it is at most 25%, even more preferably at most 20%, more preferably still at most 15%, still more preferably at most 12%.

Protein content of a biomass composition is preferably at least 20, 25, 30, 35, 40, or 45%. More preferably it is at least 30%, even more preferably at least 35%, more preferably still at least about 40%. Protein content of a biomass composition is preferably at most 70, 65, 60, 55, 50, 45, or 40%. More preferably it is at most 65%, even more preferably at most 60%, more preferably still at most 55%, still more preferably at most 50%.

It is highly preferred that the biomass composition is based on a fungal biomass, and comprises 8% to 12% (w/w, such as approximately 10 wt.-%) lipids, in addition to from about 35% to 55%, preferably 40-50 wt-% protein, such as 45% protein. More preferably it is pasteurised.

Lipids and proteins in a biomass composition can be degraded by enzymatic means like degradation using endogenous lipases and/or proteases, which can contribute to the generation of unpleasant sensory experience upon consumption, due to for example off odours. Component integrity can be further improved by for example using an antioxidant or by adjusting pasteurisation parameters and conditions as described herein.

The inventors have surprisingly found that the biomass as can be pasteurised as described herein provides a superior sensory experience. For example, as demonstrated in Example 2, the pasteurised biomass was found to offer the same olfactory experience as unpasteurised biomass produced in the same way outside of the pasteurisation step. In contrast, biomass that was pasteurised at a temperature below 75° C. was described by a tasting panel as smelling metallic, ferric, or oxidised. A preferred biomass thus has been pasteurised at a temperature of at least 75° C., preferably at least 80° C., more preferably at least 81° C., more preferably at least 82° C., still more preferably at least 83° C., even more preferably at least 84° C., more preferably still at least 85° C., most preferably about 86° C.

In Example 2, biomass that was pasteurised for more than 35 minutes was described by a tasting panel as smelling metallic, ferric, or oxidised. A preferred biomass composition thus has been pasteurised for at most 35 minutes, preferably 30 minutes, more preferably 25 minutes, even more preferably 20, 15, 10 minutes. In highly preferred embodiments, pasteurisation has taken place for at most 10, preferably 9, more preferably 8, more preferably 7, more preferably 6, more preferably 5, more preferably 4, still more preferably 3, even more preferably 2, most preferably about 1 minute.

For superior smell, excellent results can be achieved when biomass composition is pasteurised for at most 11 minutes at at least 78° C., at most 8 minutes at at least 79° C., at most 6 minutes at at least ° C., at most 5 minutes at at least 80° C., at most 4 minutes at at least 81° C., at most 3 minutes at at least 82° C., at most 2 minutes at at least 83 or 84° C., at most 1 minute at 85° C. or more, such as at 86° C.

The invention also provides a food or feed product comprising the biomass composition according to the invention. The biomass composition, preferably in pasteurised form, can be the product per se, optionally after having undergone any inspections or certifications required by local regulations. The biomass composition can also be used as an ingredient of the food or feed product. When used as an ingredient, the biomass composition is preferably a source of at least one of fiber or protein. In preferred embodiments a food product is provided, preferably for human consumption. Examples of preferred food products are meat substitutes, breakfast cereals, bread, pasta, dairy products or dairy substitutes, ice cream, chocolate, and soups. More preferred are meat substitutes and dairy substitutes. In other preferred embodiments a feed product is provided, preferably for feeding livestock or fish, more preferably livestock.

The biomass composition obtained in a process according to the invention can be used to supplement feed for a variety of different livestock animal types, including pigs, poultry, ruminant livestock as well as aquatic fish and crustacean species. For the application of the SCP as fish feed, preferably the feed is enriched with a source of omega-fatty acids fatty acids such as fish oil, or a lipid rich algae, such as Cryptocodinium cohnii, or Traustochytrium aureum. An additional advantage of the biomass composition obtained in a process according to the invention is that the acidic pH at which the SCP is produced will prevent contamination of the SCP by problematic bacteria such as E. coli, Salmonella, Bacillus cereus, Enterobacteriaceae, Listeria etc., which may be present, either dead or alive, in biomass produced in other processes.

Whereas e.g. soybean has a lysine content of appr. 6% of total amino acids, existing fungal SCPs have lysine contents of total amino acids of 8.3% for Fusarium venenatum biomass (Quorn™) or 5.6% for Pekilo protein (Paecilomyces varioti). Preferably the sum of total essential amino acids is 16%-point higher than that of soybean protein (for example such as when of Rasamsonia composticola is used) or even 25%-point higher than that of soybean protein (for example such as when Thermomucor indicae-seudatica is used). SCP from Thermomucor indicae-seudatica (e.g. strain CBS 143027) has a lysine content of more than 8.5% and even more than 10% of total amino acids and a phenylalanine contents of at least 10% of total amino acids. SCP from Thermomucor strains thus not only has a high protein content but also a high lysine and phenylalanine content. Thermomucor SCP thus has a surprisingly high nutritional value. For a food product, preferably the thermophilic fungal strain is selected from the group consisting of the strains Rasamsonia composticola strain CBS 141695, Rasamsonia emersonii CBS 143030, Thermomucor indicae-seudaticae CBS 143027, Rhizomucor miehei CBS 143029, Rhizopus sp. CBS 143160 and Rhizomucor pusillus CBS 143028.

In a further aspect the invention relates to an SCP product, preferably pasteurised, comprising protein from biomass obtainable or produced in a process as herein described above. Preferably, the SCP product comprises or consists of dried biomass with a dry matter concentration of at least 25%, 30%, 35%, 40%, 45%, 50%, 52%, 53% 54% or 55% (w/v) and which is preferably milled or extruded to an average particle size in the range of 1-3 mm. With this the product can be conveyed to pack it, or to convey it to a next processing step. The protein rich product can subsequently be dried.

Preferably, an SCP product according to the invention comprises protein from biomass of at least one thermophilic fungal strain selected from the group consisting of the strains Rasamsonia composticola strain CBS 141695, Rasamsonia emersonii CBS 143030, Thermomucor indicae-seudaticae CBS 143027 and CBS 104.75, Rhizomucor miehei CBS 143029, Rhizomucor pusillus CBS 143028, Thermoascus thermophilus CBS 528.71, Thielavia terrestris CBS 546.86, Talaromyces emersonii CBS 393.64, Thermothelomyces thermophila CBS 117.65 and Rhizopus sp. CBS 143160, of which strains CBS 141695, CBS 143030, CBS 143027, CBS 143029, CBS 143160 and CBS 143028 are preferred. The SCP product can thus be biomass or biomass cake, recovered, pressed, dried, milled and/or extruded as described hereinabove. Preferably, the (preferably pasteurised) SCP product (or the protein in the biomass) has a sum of total essential amino acids that is at least 10% higher than the sum of total essential amino acids in soybean protein. More preferably, the (pasteurised) SCP product (or the protein in the biomass) has at least one of a lysine contents of at least 8.5% of total amino acids and a phenylalanine contents of at least 10% of total amino acids.

In a further aspect the invention relates to a (preferably pasteurised) food or feed product comprising protein from biomass of at least one thermophilic fungal strain selected from the group consisting of the strains Rasamsonia composticola strain CBS 141695, Rasamsonia emersonii CBS 143030, Thermomucor indicae-seudaticae CBS 143027 and CBS 104.75, Rhizomucor miehei CBS 143029, Rhizomucor pusillus CBS 143028, Thermoascus thermophilus CBS 528.71, Thielavia terrestris CBS 546.86, Talaromyces emersonii CBS 393.64, Rhizopus sp. CBS 143160 and Thermothelomyces thermophila CBS 117.65, of which strains CBS 141695, CBS 143030, CBS 143027, CBS 143029, CBS 143160 and CBS 143028 are preferred.

The invention also encompasses the use of the biomass composition as described above in the manufacture of a food product, or as a source of at least one of fiber or protein. The use is as source that is suitable for use in the manufacture of a food product. Features and definitions are as provided elsewhere. For this use, the biomass composition is preferably pasteurised.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value more or less 5% of the value. As used herein, “subject” means any animal, preferably a mammal, most preferably a human. In preferred embodiments a subject is non-human.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

Throughout this document, when percentages are used for expressing amounts of a substance in a mixture, weight percentages are intended, unless stated otherwise or explicitly plain from context.

The present invention has been described above with reference to a number of exemplary embodiments. Modifications and alternative implementations of some parts or elements are possible, and features from embodiments can be combined where possible. All citations of literature and patent documents are hereby incorporated by reference.

### DESCRIPTION OF DRAWINGS

FIG. 1A-7D kill curve for pasteurisation of fungal biomass comprising 10% dry matter.

FIG. 1B-12D kill curve for pasteurisation of fungal biomass comprising 10% dry matter.

### EXAMPLES

### Example 1—Production of Pasteurised Single-Cell Protein

Rhizomucor pusillus strain CBS 143028 was fermented as described in WO2018/029353. Alternately, Rhizomucor pusillus strain CBS 143028 is inoculated in 200 ml of a preculture medium at pH 5.5 containing a defined mineral composition containing KCl 0.5 gr/L; KH₂PO₄ 4, Na₂HPO₄ 1.1, Citric acid 1.5 gr/L, MgSO₄.7 aq 2 gr/L, FeSO₄.7 aq 0.1 gr/L, CaCl₂.2 aq 0.1 gr/L, ZnSO₄.7 aq 0.125 gr/L, MnCl₂.4 aq 0.012, CuSO₄.5 aq 0.0016 gr/L, CoCl₂.6 aq 0.0015 gr/L, Na2B₄O₇.10 aq 0.009 gr/L KI 0.0009 gr/L, Na₂MoO₄.2 aq 0.0015 gr/L; 11 g Dextrose per l as C-source; 4 g (NH₄)₂SO₄ per l as N-source; and 7.5 g tartaric acid per l. The culture is incubated for 24 hours at 46° C., in a 1 l Erlenmeyer flask with air permeable stop with baffles, in an orbital shaker at 200 rpm. The preculture is then used to inoculate a pH 3.5 medium containing a defined mineral medium as described above comprising 77 g Dextrose per l as C-source; 1.4 g (NH₄)₂SO₄ per l as N-source and supplemented with NH3 as titrant. Olive oil is continuously being fed. More definitions are described in EP20153414.

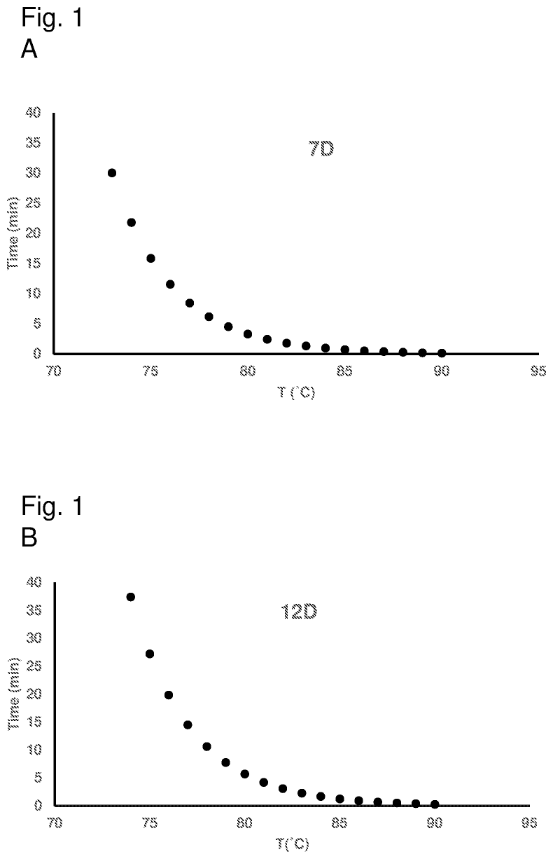

Fermentation broths, having reached a dry matter content ranging from 2 to 5 weight percent, were concentrated using a vibrating sieve to achieve a minimum of 10% (wt.) dry matter. The biomass is then pumped through an in-line heating unit comprising a pipe equipped with a steam injector. Pumping speed, pipe diameter, and temperature were configured in such a way that different residence times at different temperatures were achieved. Systematic screening of parameters allowed the generation of killing curves for various degrees of log₁₀ reduction, notably for a log₁₀ reduction of 7 (see FIG. 1A), and for a log₁₀ reduction of 12 (see FIG. 1B). Data is shown in the table below. Pasteurised samples were conveniently dried in a fluid bed drier (70° C. 10 min).

TABLE 1.1
kill kinetics using an in-line pasteurisation unit,
showing required time in minutes

| T | | | | | | |
|---|---|---|---|---|---|---|
| (° C.) | 7 D | 8 D | 9 D | 10 D | 11 D | 12 D |
| 60 | 2280.53 | 2606.32 | 2932.11 | 3257.91 | 3583.70 | 3909.49 |
| 70 | 79.27 | 90.60 | 101.92 | 113.25 | 124.57 | 135.90 |
| 71 | 57.27 | 65.45 | 73.63 | 81.81 | 89.99 | 98.17 |
| 72 | 41.45 | 47.37 | 53.29 | 59.21 | 65.13 | 71.05 |
| 73 | 30.05 | 34.35 | 38.64 | 42.93 | 47.23 | 51.52 |
| 74 | 21.83 | 24.95 | 28.07 | 31.19 | 34.31 | 37.43 |
| 75 | 15.89 | 18.16 | 20.43 | 22.70 | 24.97 | 27.24 |
| 76 | 11.59 | 13.24 | 14.90 | 16.55 | 18.21 | 19.86 |
| 77 | 8.46 | 9.67 | 10.88 | 12.09 | 13.30 | 14.51 |
| 78 | 6.19 | 7.08 | 7.96 | 8.85 | 9.73 | 10.62 |
| 79 | 4.54 | 5.19 | 5.84 | 6.48 | 7.13 | 7.78 |
| 80 | 3.33 | 3.81 | 4.29 | 4.76 | 5.24 | 5.71 |
| 81 | 2.45 | 2.80 | 3.15 | 3.50 | 3.85 | 4.20 |
| 82 | 1.81 | 2.07 | 2.32 | 2.58 | 2.84 | 3.10 |
| 83 | 1.33 | 1.52 | 1.71 | 1.91 | 2.10 | 2.29 |
| 84 | 0.99 | 1.13 | 1.27 | 1.41 | 1.55 | 1.69 |
| 85 | 0.73 | 0.83 | 0.94 | 1.04 | 1.15 | 1.25 |
| 86 | 0.54 | 0.62 | 0.70 | 0.77 | 0.85 | 0.93 |
| 87 | 0.40 | 0.46 | 0.52 | 0.58 | 0.63 | 0.69 |
| 88 | 0.30 | 0.34 | 0.39 | 0.43 | 0.47 | 0.51 |
| 89 | 0.22 | 0.26 | 0.29 | 0.32 | 0.35 | 0.38 |
| 90 | 0.17 | 0.19 | 0.21 | 0.24 | 0.26 | 0.29 |

### Example 2—Organoleptic Qualities of Pasteurised Single-Cell Protein

Pasteurised biomass obtained as described above was subjected to analysis by a testing panel. Sample 1 was pasteurised at 74° C. for 37 minutes. Sample 2 was pasteurised at 86° C. for 1 minute. Sample 3 was not pasteurised.

A testing panel of 12 people reported the following scent sensations for sample 1: metallic, ferric, oxidised, French fries, sunflower oil, raw egg. Samples 2 and 3 were generally described as odourless. Mean marks awarded for scent were 51% for sample 1, and 86% for samples 2 and 3.

### Example 3—Determination of TOTOX Values for Pasteurised Single-Cell Protein

The TOTOX value is an indication for the degree of oxidation and can be described by the following equation: (TOTOX=2PV+AV). Herein PV represents the peroxide value and AV the anisidine value. Assays for these values are known in the art. In this example, AV was determined following NEN-EN-ISO 6885:2000, PV by the certified service provider Nutrilab B.V. (Giessen, the Netherlands). Pasteurised SCP was prepared as described in Example 1, where 1000 ppm antioxidant was added at different points of the process:
A) Mixing of the antioxidant after sieving and before pasteurisation and washing.
B) Mixing of the antioxidant after sieving, pasteurisation, and washing.
C) Mixing half of the total amount of antioxidant before pasteurisation and half after washing.
D) Mixing of antioxidant after washing and before pasteurisation.

For each strategy two different antioxidants were tested, one water soluble (a mixture of carnosol and carnosic acid (rosemary extract) and flavan-3-ols (green tea extract), further comprising excipients E1520 (propylene glycol), E471 (mono- and diglycerides of edible fatty acids), and E433 (polyoxyethylene sorbitan monooleate); this mixture is available as NaturFORT 12L from Kemin) and one oil soluble (a mixture of carnosol and carnosic acid (rosemary extract) and flavan-3-ols (green tea extract), further comprising excipients E1520, E471, and E322 (Lecithin); this mixture is available as NaturFORT 15L from Kemin). Results are shown in Table 2. Before the analysis of the samples they were dried in a fluid bed dryer at 50° C. for 20 minutes and incubated at 60° C. for 3 days, mimicking a shelf life study to amplify the difference in oxidation as part of it will take place during storage.

TABLE 2
results of different antioxidant (AOX) addition strategies

| | Solubility | | | |
|---|---|---|---|---|
| Strategy | of AOX | PV | AV | TOTOX |
| A | Water | 9.4 | 21.0 | 39.8 |
| A | Oil | 7.8 | 15.7 | 31.3 |
| B | Water | 8.4 | 21.6 | 38.4 |
| B | Oil | 7.5 | 17.0 | 32.0 |
| C | Water | 7.5 | 24.0 | 39.0 |
| C | Oil | 6.8 | 36.5 | 50.1 |
| D | Water | 6.5 | 18.9 | 31.9 |
| D | Oil | 4.7 | 25.9 | 35.3 |
| No AOX | — | 9.7 | 19.2 | 38.6 |

Oil-soluble antioxidants lead to better TOTOX values.

### Example 4—Determination of TOTOX Values after Prolonged Storage

The strategy of adding the antioxidant prior to pasteurisation and washing was used for this follow-up study, where antioxidants were used at 2000 ppm. SCP was freeze dried after production, and AV, PV, and TOTOX values were determined either immediately thereafter, or after storage at 55° C. for six weeks (which represents storage at 20° C. for 1.3 years). Values were determined for SCP derived from biomass that was produced in broths with varying iron contents (by using broths with varying amounts of iron). The sample using a broth with 350 mg/kg (dry matter) iron was produced by slow growing biomass, the other three values were produced by fast growing biomass. Table 3 shows the results.

TABLE 3
TOTOX values for various conditions after prolonged storage

| iron content | | | | | | |
|---|---|---|---|---|---|---|
| (mg/kg | T = 0 | | | 6 weeks at 55° C. | | |
| dry matter) | PV | AV | TOTOX | PV | AV | TOTOX |
| 350 | 17.7 | 3.9 | 39.3 | 14.7 | 11.9 | 41.3 |
| 130 | 4.6 | 5.6 | 14.8 | 36.9 | 9 | 54.9 |
| 47.3 | 5.5 | 2.2 | 13.2 | 14 | 3.5 | 31.5 |
| 41.2 | 5.4 | 1.8 | 12.6 | 7.2 | 5.9 | 20.3 |

Reduced iron content of the pasteurised biomass leads to improved TOTOX values.

1. Method for producing a biomass composition comprising an antioxidant and biomass, comprising the steps of:
i) providing a biomass;
ii) adding an antioxidant to the biomass to obtain a biomass mixture;
iii) pasteurising the biomass mixture to obtain the biomass composition; and
iv) optionally washing the biomass composition.

2. The method according to claim 1, wherein the antioxidant is at least one of:
a) a free radical scavenger, such as carnosol; carnosic acid; rosemary extract; a flavan-3-ol such as epicatechin, epigallocatechin, epigallocatechin gallate (EGCG), epicatechin gallate; a flavonoid such as kaemferol, quercetin, or myricetin; green tea extract; butylated antioxidants such as butylated hydroxytoluene (BHT), butylated hydroxyanisole, or tert-butylhydroquinone (TBHQ); gallic acid; a gallate ester such as propyl gallate; carvone; a caffeate ester such as methyl caffeate, ethyl caffeate, caffeic acid phenethyl ester (CAPE), or rosmarinic acid; spearmint extract; a vitamin E such as a tocopherol or a tocotrienol; a vitamin A such as retinol, retinoic acid, or retinal; a provitamin A such as a carotenoid, β-carotene, lutein, zeaxanthin, or echinenone; a quinolone-based antioxidant such as ethoxyquin; or seaweed extract; or
b) an oxygen reducer, such as ascorbic acid or acerola extract; or
c) a chelator, such as citric acid or EDTA.

3. The method according to claim 1, wherein the obtained biomass composition has a TOTOX value of under 50, and wherein the TOTOX value preferably remains under 50, for at least 6 weeks.

4. The method according to claim 1, wherein the biomass is derived from a fermentation broth, wherein the fermentation broth preferably comprises a biomass dry matter content of at most 6%, more preferably of at most 4%, or
wherein the fermentation broth is sieved to obtain the biomass.

5. The method according to claim 4, wherein the biomass comprises at most 500 mg/kg dry matter iron.

6. The method according to claim 1, wherein the pH of the biomass in step iii) is at most 4.5.

7. The method according to claim 1, wherein the biomass comprises at least 7% dry matter.

8. The method according to claim 1, wherein the biomass is a biomass derived from a fungal strain, wherein the fungal strain is preferably a strain of a fungal genus selected from the group consisting of Rasamsonia, Talaromyces, Penicillium, Acremonium, Humicola, Paecilomyces, Chaetomium, Rhizomucor, Rhizopus, Thermomyces, Myceliophthora, Thermoascus, Thielavia, Mucor, Stibella, Melanocarpus, Malbranchea, Dactylomyces, Canariomyces, Scytalidium, Myriococcum, Corynascus, and Coonemeria.

9. The method according to claim 1, wherein the pasteurisation is performed in an in-line heating unit that preferably comprises a pipe heater, a heating block, or a steam infusion element, and wherein the in-line heating unit optionally comprises a mixing element such as a static mixer.

10. The method according to claim 1, wherein the pasteurisation is performed for at most 45 minutes.

11. The method according to claim 1, wherein pasteurisation is performed at a temperature of at least 70° C.

12. The method according to claim 1, wherein antioxidant is only added during step ii).

13. Biomass composition comprising an antioxidant and biomass, obtained by a method as described in claim 1.

14. Food or feed product comprising the biomass composition according to claim 13.

15. (canceled)

16. The biomass composition according to claim 13, wherein the biomass comprises from about 6% to 12% lipids and from about 35% to 55% proteins, based on dry weight.

17. The method according to claim 8, wherein the fungal genus is Rhizomucor.

18. The method according to claim 8, wherein the fungal the strain is Rhizomucor pusillus.

19. The method according to claim 18, wherein the Rhizomucor pusillus strain is CBS 143028, or a strain that is a single colony isolate or a derivative thereof.
